(19) United States
(12) Patent Application Publication
(10) Pub. No.: US 2026/0226560 A1
(43) Pub. Date: Aug. 6, 2026

(54) NONDISCRIMINATORY DETECTION OF RARE VARIANTS

(71) Applicant: Bio-Rad Laboratories, Inc., Hercules, CA (US)

(72) Inventors: Ronald Lebofsky, Sceaux (FR); Mona Shahbazian, Hercules, CA (US); Mina Rostamza, Hercules, CA (US); Surbhi Jain, Mountain House, CA (US); Mai Ho, Oakland, CA (US)

(21) Appl. No.: 19/529,903

(22) Filed: Feb. 4, 2026

Related U.S. Application Data
(60) Provisional application No. 63/754,287, filed on Feb. 5, 2025.

(51) Int. Cl. C12Q 1/6886; C12Q 1/6851

(52) U.S. Cl. CPC C12Q 1/6886; C12Q 1/6851; C12Q 2600/156

(57) ABSTRACT

Methods for the non-discriminatory detection of variants by digital PCR (dPCR) with a blocker for the non-target molecules. A set of probes produce a detectable signal upon the amplification of one or more variants. When the detectable signal is detected, the presence of at least one the variants is indicated. The methods need not indicate the identity of the variant. Methods of detecting the cis or trans configuration of variants using a blocker are provided. Probes detect at least a first variant and a second variant with a first signal and a second signal, respectively. Detection of only one signal from each partition indicates a trans configuration, while detection of both signals indicates cis. In both embodiments, the blocker binds to the non-target molecule and prevents amplification of the non-target from contributing a detectable signal, inhibiting signal from a wild-type sequence.

### TECHNICAL FIELD

The disclosure relates to the detection of molecular targets, including the relative location of multiple mutations.

### BACKGROUND

Numerous clinical and research methods involve the detection of specific nucleic acids. For example, cancer is typically associated with certain mutations in tumor DNA and the ability to detect those mutations can be informative of the presence or progress of the cancer or the success of a treatment. In theory, after a patient is treated to remove a tumor, the success of the treatment can be evaluated by performing an assay to detect tumor DNA. It is understood that tumor cells and cell-free tumor DNA can be found circulating in blood. Detecting such circulating tumor nucleic acid in a laboratory assay would be a valuable diagnostic tool.

Unfortunately, circulating tumor DNA is only a minor fraction of a blood draw, which makes it a challenge to detect. Such a sample also includes abundant DNA from non-tumor cells. Additionally, such tumor DNA may have a diverse variety of different genetic mutations, each of which may be clinically significant. Similar challenges arise in pathogen detection, agriculture, and other fields of endeavor. For example, there is increasing interest in assaying wastewater to monitor patterns of viral spread in communities. However, in such a sample, the target of interest may be present only in minor fractions and may also have genetic diversity among those viral nucleic acids that are present. For example, a wastewater system that serves a large metropolitan region may have trace amounts of viral nucleic acid that are variously derived from different variants of a virus that is spreading through the region.

Some approaches to detecting such nucleic acids of interest have involved capture and amplification of those targets by, for example, polymerase chain reaction (PCR). However, PCR based assays may not be fully satisfactory for certain samples, particularly where the target of interest is only present in small quantities, especially if among abundant quantities of similar, but less-informative molecules.

Further, in many contexts, the detection of a specific mutation is not required. A practitioner might wish to screen a sample for several variants without interest as to which variant or variants are present. For example, in the analysis of blood samples for cancer mutations, there may be multiple mutations of a single gene which are known to play a role in cancer etiology. The detection of any of these mutations may provide useful information for further screening of the patient.

### SUMMARY

The present disclosure provides methods for the detection of molecular targets such as nucleic acid variants of non-target molecules with blocker molecules that inhibit the amplification and/or detection of non-target molecules in a sample. Such detection may be achieved using nucleic acid amplification techniques, such as digital PCR (dPCR). For dPCR , a sample is divided into a large number (e.g., tens of thousands) of aqueous partitions and a PCR reaction is conducted in each partition with primers and probes that generate amplicons and give a detectable signal when a target variant is present in a partition.

One embodiment of the present disclosure provides nondiscriminatory detection from a plurality of potential variants. A set of probes is utilized in a PCR reaction which is designed to detect at least two variants from the plurality of variants. Upon amplification of one of the at least two variants, one or more of the probes produces a detectable signal. The presence or absence of the signal indicates the presence or absence of at least one of the plurality of variants. The above process is nondiscriminatory in that the detection of a signal does not provide indication of which variant is present.

Additional embodiments of the disclosure provide methods of detecting the relative location of two or more variants. Variants may be present on the same allele (cis configuration) or on different alleles (trans configuration). These embodiments utilize a set of probes including at least a first probe that produces a first detectable signal upon amplification of a first variant and a second probe that produces a second detectable signal upon amplification of a second variant. Partitioning of the sample and amplification are performed according to known dPCR techniques. When reading or analyzing the results, the presence of both the first detectable signal and the second detectable signal in one or more partitions of the plurality of partitions indicates that the first variant and the second variant are present in a cis configuration, and the presence of only the first detectable signal in a first subset of partitions of the plurality of partitions and the presence of only the second detectable signal in a second subset of partitions of the plurality of partitions indicates that the first variant and the second variant are present in a trans configuration.

In some embodiments, the detectable signal is produced by a detectable label. Two probes providing the same signal may utilize the same label or different labels. In most embodiments, the detectable label is optically detected (i.e., an optically detectable label). In some embodiments, the optical label may be a fluorescent label (e.g., a fluorophore or a fluorescent protein), a colorimetric label, or a chemiluminescent label. In some embodiments, the detectable signal comprises more than one fluorescent signal detected in more than one optical detection channel.

Some embodiments of this disclosure utilize mediator hydrolysis probes. These hydrolysis probes differ from fluorescent hydrolysis probes. The mediator hydrolysis probes comprise a 3’ end sequence that hybridizes to a target variant and a cleavable 5’ end sequence that can hybridize to a universal reporter. Upon amplification of the variant, the 5’ end sequence is cleaved and hybridizes with the universal reporter to provide the detectable signal. The universal reporter may include a detectable label as identified above.

The set of probes includes at least one probe which provides a detectable signal upon amplification of at least one of the plurality of variants. The set of probes may contain 1 or more, 2 or more, 5 or more, 10 or more, 15 or more, 20 or more, or 25 or more probes. In the case of multiple probes within the set of probes, each probe may provide a detectable signal upon amplification of a corresponding variant. In other embodiments, a single probe may provide a detectable signal upon the amplification of 1-25 or more (e.g., any of 1, 2, 3, 4, 5, 6, 7, 8 ,9, 10, 11, 12, 13, 14, 15, 16, 17, 18, 19, 20, 21, 22, 23, 24, 25, or more) different variants. For those embodiments relying on a first detectable signal and a second detectable signal, the set of probes would include at least two probes, one to provide each signal.

Detection of rare targets is promoted by the use and inclusion of a blocker. The blocker is a molecule or molecular species that binds to or suppresses at least one non-target molecule from contributing a detectable signal from the partitions. The blocker typically does not include a detectable label.

For example, the blocker can be an oligonucleotide that is perfectly complementary to a non-target nucleic acid, or a protein, or an RNA-guided binding protein. The blocker will bind to the non-target molecule and to any amplicon copies thereof. The blocker may inhibit or displace any probe from binding to those molecules, or the blocker may impede, block or displace one of the primers from binding to those molecules, thus inhibiting amplification and detection of the non-target. Due to these effects, the blocker removes the non-target molecule from the dPCR readout.

The combination of multiplex probes and blockers work particularly well together to prevent wild-type sequences from being read in a dPCR assay for rare mutations or variants. With methods of the invention, a plurality of cancer-associated mutations can be reliably detected in a multiplex digital PCR assay while wild-type alleles are blocked from being detected/read.

In support of readouts such as dPCR, the methods involve portioning the sample and reagents into partitions. The partitions may be droplets, wells (e.g., microwells) in a plate or chip (e.g., a microfluidic chip), or other fluid portioning structure. Methods preferably include diluting the sample so that each partition receives a limited number of target molecules, such as zero, one, two, sometimes three, and a very small number of four or more. The dilution can be calculated so that a majority of partitions receive a target number (e.g., zero or one) of targets. Each target molecule will serve as a template for generation of amplicons in the presence of fluorescence probes. Methods may include thermocycling the droplets within a reaction tube, chamber, or well of a plate or may include isothermal amplification. Exemplary probes include an oligonucleotide backbone that anneals in a sequence specific manner to a target of that probe, plus a fluorophore and a quencher. During amplification with polymerase, exonuclease activity of the polymerase digests the oligonucleotide backbone of any bound probe, separating the fluorophore from the quencher, allowing the fluorophore to fluoresce during a readout step. To read fluorescence, methods may include flowing the droplets (e.g., one-at-a-time) past a detector (and optionally a light source for excitation of fluorophores) or imaging the partitions (e.g., imaging droplets or wells such as microwells).

The amplification step may include thermocycling, using reagents that include PCR primers and dNTPs. In some embodiments, the probes include fluorescent hydrolysis probes that each anneal to a sequence of one of the variants. The blocker may include an oligonucleotide that anneals to a wild-type version of the variants and inhibits amplification of wild type sequences. In some cases, the blocker may include an oligonucleotide that anneals to a wild-type version of the variants and prevents one or more primers and/or fluorescent probes from binding to the wild-type version or any amplicons thereof. In some cases, the blocker may include an oligonucleotide that anneals to a wild-type version of the variants and prevents one or more primers from binding to the wild-type version or any amplicons thereof. In some embodiments, the blocker prevents binding of a forward primer to the wild-type version or any amplicons thereof. In some embodiments, the blocker prevents binding of a reverse primer to the wild-type version or any amplicons thereof. The blocker may include other features (e.g., one or more locked-nucleic acids in the oligonucleotide) or other molecular strategies. For example, in some embodiments, the blocker is a binding protein (such as an RNA-guided binding protein) that binds to the non-target molecule and prevents the amplification.

The detecting step may include detecting signal (e.g., fluorescence) from the partitions. The reporting step may include reporting the presence or absence of the plurality of variants in the sample. In some embodiments, the method is used to detect mutant versions of, or variants of, a gene.

In certain embodiments, the variants are present in tumor DNA in the sample, and the method (i) includes isolating circulating tumor DNA (ctDNA) from the sample, or (ii) isolating circulating tumor cells (CTCs) from the sample and purifying DNA from the CTCs.

Preferably, each of the variants is targeted with a respective probe or probe combination that includes a sequence specific to that variant. In some embodiments, a single probe targets more than one variant (e.g., any of 2, 3, 4, 5, 6, 7, 8 ,9, 10, 11, 12, 13, 14, 15, 16, 17, 18, 19, 20, 21, 22, 23, 24, 25, or more, different variants).

The variants may be non-wild type variants of a wild-type gene, where the variants may be of, e.g., clinical significance, and the blocker is used to suppress the wild-type out of digital PCR readouts. For example, the blocker may inhibit detection of a wild-type sequence of a gene where each of the variants include a mutated portion of the gene (i.e., that includes a mutation, relative to the wild-type sequence).

Exemplary genes may include ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEB PA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (Cllorf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLDI, POLE, PPARG, PPP2R1A, PPP2R2A, PRDM1, PRKAR1A, PRKCI, PTCHI, PTEN, PTPN11, PTPRO, QKI, RAC1, RAD21, RAD51, RAD51B, RAD51C, RAD51D, RAD52, RAD54L, RAFI, RARA, RBI, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSCI, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTEA-4, dMMR, EGFR, ERBB1, ERBB2, FGFR1-3, FLT3, GD2, HDAC, HER1, HER2, HR, IDH2, IL-ip, IL-6, IL-6R, JAK1, JAK2, JAK3, KIT, KRAS, MEK, MET, MSI-H, mTOR, PARP, PD-1, PDGFR, PDGFRa, PDGFRp, PD-L1, PI3K5, PIGF, PTCH, RAF, RANKL, RET, R0S1, SLAMF7, VEGF, VEGFA, or VEGFB, In some embodiments, the gene is BRAF, EGFR, KRAS, NRAS, PIK3CA, or ESR1. In certain embodiments, the sample includes tumor DNA from a subject, the blocker suppresses detection of wild-type sequences from non-tumor DNA from the subject, and detection of the plurality of variants (presence or absence) shows the presence or grade of a tumor in the subject.

The present disclosure also provides kits and articles of manufacture for detecting at least one variant of a plurality of variants of a non-target nucleic acid in a sample, comprising: nucleic acid amplification reagents including a set of primers to amplify at least a portion of each of the plurality of variants; a set of probes that detects at least two variants of the plurality of variants, wherein the set of probes produces a first detectable signal upon amplification of one or more of the at least two variants; and a blocker that inhibits amplification of the non-target nucleic acid. In some embodiments, the kits or articles of manufacture may be used for detecting at least one variant in a sample according to any of the methods described herein, e.g., in a nondiscriminatory manner. In some embodiments, the sample comprises the non-target nucleic acid and potentially comprises one or more of the plurality of variants of the non-target nucleic acid.

The present disclosure also provides kits and articles of manufacture for detecting a cis or trans configuration of at least two variants of a plurality of variants of a non-target nucleic acid in a sample, comprising: nucleic acid amplification reagents including a set of primers to amplify at least a portion of each of the plurality of variants; a set of probes that detect at least two variants of the plurality of variants, wherein the set of probes comprises: at least a first probe that is specific for a first variant of the plurality of variants and produces a first detectable signal upon amplification of the first variant, and at least a second probe that is specific for a second variant of the plurality of variants and produces a second detectable signal upon amplification of the second variant; and a blocker that inhibits amplification of the non-target nucleic acid. In some embodiments, the kits or articles of manufacture may be used for detecting a cis or trans configuration of at least two variants in a sample according to any of the methods described herein. In some embodiments, the sample comprises the non-target nucleic acid and potentially comprises one or more of the plurality of variants of the non-target nucleic acid.

The present disclosure also provides methods for detecting presence, progression, response to a treatment, or grade of a cancer in a subject, based on detecting at least one variant of a plurality of variants of a non-target nucleic acid in a sample from the subject according to any of the detection methods provided herein, e.g., in a nondiscriminatory manner. The present disclosure also provides methods for detecting presence, progression, response to a treatment, or grade of a cancer in a subject, based on detecting a cis or trans configuration of at least two variants of a non-target nucleic acid in a sample from the subject according to any of the detection methods provided herein. In some embodiments, the subject is a human.

### BRIEF DESCRIPTION OF THE DRAWINGS

FIGS. 1-7 illustrate nondiscriminatory multiplexed digital PCR detection of seven insertions in the Epidermal Growth Factor Receptor (EGFR) gene combined with use of a wild type blocker to suppress signal from background wild type DNA.

FIG. 1 shows the results for a sample containing c.2303_2311dup (V769_D770insASV).

FIG. 2 shows the results for a sample containing c.2307_2308insGCCAGCGTG (S768_D770dup).

FIG. 3 shows the results for a sample containing c.2310_2311insGGT (D770_N771insG).

FIG. 4 shows the results for a sample containing c.2311_2319dup (N771_H773dup),

FIG. 5 shows the results for a sample containing c.2314_2319dup (P772_H773dup),

FIG. 6 shows the results for a sample containing c.2319-2320insAAACCCCAC (H773_V774insNPH), and

FIG. 7 shows the results for a sample containing c.2317_2319dup (H773dup).

FIGS. 8-14 illustrate nondiscriminatory multiplexing of a digital PCR assay for seven deletions in the EGFR gene combined with use of a wild type blocker to suppress signal from background wild type DNA.

FIG. 8 shows the results for a sample containing c.2235_2249del (E746_A750del).

FIG. 9 shows the results for a sample containing c.2236_2250del (E746_A750del).

FIG. 10 shows the results for a sample containing c.2240_2257del (L747_P753delinsS).

FIG. 11 shows the results for a sample containing c.2239_2247del (L747_E749del).

FIG. 12 shows the results for a sample containing c.2239_2258delinsCA (L747_P753delinQ).

FIG. 13 shows the results for a sample containing c.2239_2248delinsC (L747_P750delinsP).

FIG. 14 shows the results for a sample containing c.2238_2248delinsGC (L747_A750delinsP).

FIGS. 15-17 illustrate nondiscriminatory digital PCR multiplexing for three amino acid substitutions in the EGFR gene combined with use of a wild type blocker to suppress signal from background wild type DNA.

FIG. 15 shows the results for a sample containing c.2156G>C (G719A).

FIG. 16 shows the results for a sample containing c.2155G>A (G719S).

FIG. 17 shows the results for a sample containing c.2155G>T (G719C).

FIGS. 18-24 illustrate digital PCR multiplexing for variants in the EGFR gene to ascertain if two nearby amino acid changes (C797S and T790M) arise from mutations in the same (cis) or different (trans) alleles.

FIG. 18 shows the results for a sample containing only T790M.

FIG. 19 shows the results for a sample containing only C797S-T>A.

FIG. 20 shows the results for a sample containing only C797S-G>C.

FIG. 21 shows the results for a sample containing T790M and C797S-T>A present in the same allele (cis).

FIG. 22 shows the results for a sample containing T790M and C797S-G>C present in the same allele (cis).

FIG. 23 shows the results for a sample containing T790M and C797S-T>A present on different alleles (trans).

FIG. 24 shows the results for a sample containing T790M and C797S-G>C present on different alleles (trans).

### DETAILED DESCRIPTION

The invention provides methods for detecting targets of interest and in particular provides for the use of digital PCR based detection for the nondiscriminatory detection of one or more of a plurality of genetic targets in a single readout operation. Some embodiments are useful for the detection of whether two genetic targets are present on the same allele in a cis configuration or different alleles in a trans configuration. Additionally, methods of the invention make use of a blocker that suppresses or inhibits the ability of a non-target nucleic acid sequence to generate any signal in a digital PCR assay. The blocker is particularly useful where several sequences of interest are related but present in relatively small quantity as compared to a predominant sequence (e.g., a wild type). A digital PCR assay of the disclosure may be performed using reporters that give the same signals for any one of the plurality of variants of interest while a blocker minimizes any signal from the wild-type sequence.

For example, the digital PCR assay may involve amplification of nucleic acids from a sample in aqueous partitions with primers. The sample may include some number of variants of interest as well as an abundant number of copies of the wild-type or non-target sequence. In such a case, those templates are genetic homologs. Those templates (the wild-type plus the variants) are amplified using the primers. In some embodiments, the primers amplify both wild-type or non-target as well as variant templates. In some embodiments, allele-specific primers may be used. In such cases, a variant-specific primer or primer pair specifically amplifies one or more variant templates. The partitions include detectably labeled probes for the variants of-interest. Because of the homology among those templates, some variant-specific probes may anneal to the wild-type template, or amplicons thereof. Were such binding to happen, the wild-type template would generate a signal that would be detected in the final readout. The invention uses a blocker to inhibit signal from the wild-type template. For example, the blocker may be an oligonucleotide that binds specifically to the wild-type template. To maximize its effect as a blocker, the blocker may include a wild-type specific base at one end, may include one or more locked nucleic acids, may be designed against a GC-rich stretch of the wild-type template, or other such features or combinations thereof. The blocker may include a fully wild type sequence. The blocker may include a blocking moiety at one end to prevent extension by a DNA polymerase. For example, the blocker may include a blocking moiety at the 3’ end to prevent extension by DNA polymerase. Such blocking moieties may include modifications such as minor groove binders, inverted DNA nucleotides, 3-carbon spacers, modified or unmodified nucleotides (e.g., 1, 2, 3, 4, 5, 6, 7, 8, 9, 10, or more nucleotides) that do not pair with the template, or other moieties known in the art.

In some embodiments, during PCR with, e.g., fluorescent hydrolysis probes, the blocker anneals to wild-type sequences and inhibits amplification and non-specific binding by one or more primers and/or variant-specific probes. In other embodiments, during PCR with, e.g., fluorescent hydrolysis probes, the blocker may anneal to template and/or amplicons thereof and cannot be displaced by variant-specific probes. In other embodiments, during PCR the blocker may anneal to wild type or non-target template and/or amplicons thereof and cannot be displaced by one or more primers. In other embodiments, during PCR the blocker may outcompete one or more primers for annealing to wild type or non-target template and/or amplicons thereof. By virtue of the blocker, each aqueous partition generates substantial signal only when one of the probe-targeted variants is in that partition.

By virtue of its inhibition of detectable signal from a dominant species, the blocker facilitates the successful multiplex detection of the minor species in the sample. That is, the presence or absence of any of multiple (e.g.,1, 2, 3, 4, 5, 6, 7, 8, 9 ,10, 11, 12, 13, 14, 15, 16, 17, 18, 19, 20, 21, 22, 23, 24, 25, or more) variants can be detected without consideration of which variant is present in part because the dominant wild-type has been suppressed. While stated here in terms of mutant variants and a wild-type sequence, methods of the invention promote successful multiplex detection in numerous contexts and biological situations, particularly where there are multiple minor (in quantity) targets of interest in a sample that includes a predominant target. For example, multiplex detection according to methods of the disclosure may use a blocker to suppress healthy, non-tumor sequences when assaying for tumor mutations. Methods may use a blocker to suppress signal from maternal DNA when assaying for circulating fetal nucleic acids. Methods of the invention may be used for the multiplexed detection of pathogenic strains of a bacteria within a sample dominated by a benign strain. Methods of the invention may be used to detect the presence of any of multiple variants of a virus (e.g., in wastewater or sewage) while blocking detection of a variant for which the public has received an effective vaccine.

### Nondiscriminatory Detection

Some embodiments of the disclosure relate to a nondiscriminatory detection method. The method comprises partitioning into a plurality of partitions an aqueous sample that comprises a non-target nucleic acid and potentially comprises one or more of a plurality of variants of the non-target nucleic acid. The partitions include nucleic acid amplification reagents including a set of primers that amplify at least a portion of each of the plurality of variants if present, a set of probes that detects at least two variants of the plurality of variants, and a blocker that inhibits amplification of the non-target nucleic acid.

The partitions are subjected to conditions that promote nucleic acid amplification, so that the set of probes produces a first detectable signal upon amplification of one or more of the at least two variants (when present). Accordingly, the presence or absence of at least one variant of the plurality of variants is detected in the sample based on detecting the first detectable signal from one or more of the partitions.

In some embodiments, the method comprises detecting the presence of at least one variant of the plurality of variants in the sample based on detecting the first detectable signal. For these embodiments, the presence of the variant(s) is indicated by detecting the first detectable signal from one or more of the partitions.

In some embodiments, the detecting step does not discriminate between different variants of the plurality of variants. Stated differently, in some embodiments, the detecting step does not determine which specific variant or variants are present in the sample.

Some embodiments further comprise reporting the presence of at least one of the plurality of variants in the sample based on detection of the first detectable signal. For these embodiments, the presence of at least one variant of the plurality of variants in the sample may be reported without reporting which specific variant or variants of the plurality of variants are present in the sample.

Each probe used in this method may be selected to potentially detect a single variant of the plurality of variants, or at least two variants (e.g., any of 2, 3, 4, 5, 6, 7, 8, 9 ,10, 11, 12, 13, 14, 15, 16, 17, 18, 19, 20, 21, 22, 23, 24, 25, or more) of the plurality of variants. In some embodiments, each probe of the set of probes comprises a detectable label that produces the first detectable signal upon amplification of its corresponding variant. Further, in some embodiments, at least two probes of the set of probes comprise the same detectable label that produces the first detectable signal. Alternatively, in some embodiments, at least two different probes of the set of probes comprise different detectable labels that each produce the first detectable signal.

The detectable label may be an optically detectable label such as a fluorescent label, a colorimetric label, or a chemiluminescent label. In some embodiments, the fluorescent label is a fluorophore or a fluorescent protein. The fluorescent label may be selected from carboxyfluorescein (FAM), hexachlorofluorescein (HEX), cyanine5 (CY5), Quasar 670, cyanine5.5 (CY5.5), Quasar 705, TYE 705, 5-carboxy-X-rhodamine (5-ROX), Cal Fluor Red 590, TYE665, a fluorescent oligonucleotide dye with 594 nm absorption, and ATTO590.

Accordingly, in some embodiments, the first detectable signal is a fluorescent signal, a colorimetric signal, or a chemiluminescent signal.

When the first detectable signal is a fluorescent signal it may be detectable in a first optical detection channel. Further, the first detectable signal may comprise a first fluorescent signal detectable in a first optical detection channel and a second fluorescent signal detectable in a second optical detection channel.

For nondiscriminatory detection, the set of probes may comprise any number of probes. In some embodiments, the set of probes comprises 1-25 probes. In some embodiments, the set of probes comprises 25 or more probes.

In some embodiments, each probe of the set of probes produces the first detectable signal upon amplification of its corresponding variant. Further, in some embodiments, each probe of the set of probes is specific to a different variant of the plurality of variants, and each probe produces the first detectable signal upon amplification of its corresponding variant. In some specific embodiments, the set of probes may comprise one probe that is specific to 2-25, or more, e.g., any of 2, 3, 4, 5, 6, 7, 8, 9 ,10, 11, 12, 13, 14, 15, 16, 17, 18, 19, 20, 21, 22, 23, 24, 25, or more, different variants of the plurality of variants, and the one probe produces the first detectable signal upon amplification of one or more of said different variants.

### Cis/trans Detection

Additional embodiments of the disclosure relate to a detection method for identifying cis/trans configurations. The method comprises partitioning an aqueous sample that comprises a non-target nucleic acid and potentially comprises a plurality of variants of the non-target nucleic acid into a plurality of partitions. The partitions include nucleic acid amplification reagents including a set of primers that amplify at least a portion of each of the plurality of variants, a set of probes that detects at least two variants of the plurality of variants, and a blocker that inhibits amplification of the non-target nucleic acid. The set of probes comprises at least a first probe that is specific for a first variant of the plurality of variants and produces a first detectable signal upon amplification of the first variant, and at least a second probe that is specific for a second variant of the plurality of variants and produces a second detectable signal upon amplification of the second variant. The partitions are subjected to conditions that promote nucleic acid amplification.

A cis or trans configuration of at least the first variant and the second variant in the sample is detected by detecting the presence or absence of the first detectable signal and the second detectable signal in one or more partitions of the plurality of partitions. The presence of both the first detectable signal and the second detectable signal in one or more partitions of the plurality of partitions (e.g., in a greater number of partitions than expected by random partitioning, for example, based on a Poisson distribution) indicates that the first variant and the second variant are present in the sample in a cis configuration. And the presence of only the first detectable signal in a first subset of partitions of the plurality of partitions and presence of only the second detectable signal in a second subset of partitions of the plurality of partitions indicates that the first variant and the second variant are present in the sample in a trans configuration. As used in this regard, a cis configuration identifies two variants which are present on the same allele, whereas a trans configuration identifies mutations which are present on different alleles.

In specific embodiments, the first probe comprises a first detectable label that produces the first detectable signal upon amplification of the first variant and the second probe comprises a second detectable label that produces the second detectable signal upon amplification of the second variant. In some embodiments, the first and second detectable labels are optically detectable labels (e.g., a fluorescent label, a colorimetric label, or a chemiluminescent label). A fluorescent label may be a fluorophore or a fluorescent protein. The fluorescent label may be selected from carboxyfluorescein (FAM), hexachlorofluorescein (HEX), cyanine5 (CY5), Quasar 670, cyanine5.5 (CY5.5), Quasar 705, TYE 705, 5-carboxy-X-rhodamine (5-ROX), Cal Fluor Red 590, TYE665, a fluorescent oligonucleotide dye with 594 nm absorption, and ATTO590.

In some embodiments, the first detectable signal is a first fluorescent signal and the second detectable signal is a second fluorescent signal. For these embodiments, the first detectable signal may be a first fluorescent signal detectable in a first optical detection channel and the second detectable signal may be a second fluorescent signal detectable in a second optical detection channel. Alternatively, the first detectable signal may be a first fluorescent signal detectable at a first intensity in a first optical detection channel and the second detectable signal may be the first fluorescent signal detectable at a second intensity different from the first intensity in the first optical detection channel. Alternatively, the first detectable signal may comprise a first fluorescent signal detectable in a first optical detection channel and a second fluorescent signal detectable in a second optical detection channel, and/or the second detectable signal may comprise a third fluorescent signal detectable in a third optical detection channel and a fourth fluorescent signal detectable in a fourth optical detection channel.

For cis/trans detection, the set of probes may comprise 2-25, or more, e.g., any of 2, 3, 4, 5, 6, 7, 8, 9 ,10, 11, 12, 13, 14, 15, 16, 17, 18, 19, 20, 21, 22, 23, 24, 25, or more, probes. In some embodiments, each probe of the set of probes produces either the first detectable signal or the second detectable signal upon amplification of its corresponding variant. Accordingly, for some embodiments, each probe of the set of probes is specific to a different variant of the plurality of variants, and each probe produces either the first detectable signal or the second detectable signal upon amplification of its corresponding variant.

In some embodiments, the first probe is specific to 1-25, or more, e.g., any of 1, 2, 3, 4, 5, 6, 7, 8, 9 ,10, 11, 12, 13, 14, 15, 16, 17, 18, 19, 20, 21, 22, 23, 24, 25, or more, different variants of the plurality of variants, and the first probe produces the first detectable signal upon amplification of one or more of said different variants. Similarly, in some embodiments, the second probe is specific to 1-25, or more, e.g., any of 1, 2, 3, 4, 5, 6, 7, 8, 9 ,10, 11, 12, 13, 14, 15, 16, 17, 18, 19, 20, 21, 22, 23, 24, 25, or more, different variants of the plurality of variants, and the second probe produces the second detectable signal upon amplification of one or more of said different variants.

For some embodiments, the set of probes comprises at least a third probe that is specific to a third variant and produces the first detectable signal upon amplification of the third variant, and/or the set of probes comprises at least a fourth probe that is specific to a fourth variant and produces the second detectable signal upon amplification of the fourth variant.

In some embodiments, the set of probes comprises 1-25, or more, e.g., any of 1, 2, 3, 4, 5, 6, 7, 8, 9 ,10, 11, 12, 13, 14, 15, 16, 17, 18, 19, 20, 21, 22, 23, 24, 25, or more, different probes specific to a different variant of the plurality of variants that each produce the first detectable signal upon amplification of its corresponding variant, and/or the set of probes comprises 1-25, or more, e.g., any of 1, 2, 3, 4, 5, 6, 7, 8, 9 ,10, 11, 12, 13, 14, 15, 16, 17, 18, 19, 20, 21, 22, 23, 24, 25, or more, different probes specific to a different variant of the plurality of variants that each produce the second detectable signal upon amplification of its corresponding variant.

Probes of any suitable type are encompassed by the scope of this disclosure. In some embodiments, probes of the set of probes may comprise molecular beacon probes, mediator hydrolysis probes, or fluorescent hydrolysis probes.

### Mediator Hydrolysis Probes

For some embodiments of the disclosure, the set of probes comprises one or more mediator hydrolysis probes. The mediator hydrolysis probes of the disclosure comprise a 3’ end sequence that hybridizes to a variant, and a cleavable 5’ end sequence that does not hybridize to the variant but that hybridizes to a universal reporter present in the partitions. The 5’ end sequence is cleaved from the probe during amplification of the variant. Subsequently, the universal reporter produces a detectable signal upon hybridizing to the cleaved 5’ end sequence. Exemplary mediator hydrolysis probes and universal reporters that may be used in the methods of the disclosure are described in, e.g., Faltin et al., Clin Chem. 2012 Nov;58(11):1546-56; WO2012096430; WO2014104818, each incorporated by reference herein.

Accordingly, the universal reporter comprises a detectable label that produces the detectable signal. As above, in some embodiments, the detectable label is an optically detectable label, including a fluorescent label, a colorimetric label, or a chemiluminescent label. The fluorescent label may be a fluorophore or a fluorescent protein.

In some embodiments, each of the set of probes is a mediator hydrolysis probe. Accordingly, in some embodiments, the set of probes comprises a first mediator hydrolysis probe specific to a first variant and a second mediator hydrolysis probe specific to a second variant. For these embodiments, the universal reporter produces the first detectable signal upon hybridizing to the cleaved 5’ end sequence of the first mediator hydrolysis probe and/or of the second mediator hydrolysis probe.

The universal reporter may comprise a detectable label that produces the first detectable signal. Again, the detectable label may be a fluorescent label, a colorimetric label, or a chemiluminescent label. The fluorescent label may be a fluorophore or a fluorescent protein.

For these embodiments, the first detectable signal is a fluorescent signal, a colorimetric signal, or a chemiluminescent signal. Accordingly, in some embodiments, the first detectable signal is a fluorescent signal detectable in a first optical detection channel. While in other embodiments, the first detectable signal may comprise a first fluorescent signal detectable in a first optical detection channel and a second fluorescent signal detectable in a second optical detection channel.

For methods of cis/trans detection, the first probe may be a first mediator hydrolysis probe specific to the first variant and the second probe may be a second mediator hydrolysis probe specific to the second variant. For these embodiments, a first universal reporter present in the partitions produces the first detectable signal upon hybridizing to the cleaved 5’ end sequence of the first mediator hydrolysis probe, and/or a second universal reporter present in the partitions produces the second detectable signal upon hybridizing to the cleaved 5’ end sequence of the second mediator hydrolysis probe.

Accordingly, in some embodiments, the first universal reporter comprises a first detectable label that produces the first detectable signal upon hybridizing to the cleaved 5’ end sequence of the first mediator hydrolysis probe; and/or the second universal reporter comprises a second detectable label that produces the second detectable signal upon hybridizing to the cleaved 5’ end sequence of the second mediator hydrolysis probe. The first and second detectable labels are defined as above.

### Blocker

As identified above, the blocker prevents amplification and/or detection of a non-target molecule. Accordingly, in most embodiments, the blocker does not comprise a detectable label. For most embodiments, the blocker comprises an oligonucleotide, a protein, or an RNA-guided binding protein.

As described above, the blocker may block binding of a primer to the non-target nucleic acid. For these embodiments, the blocker may block binding of the 3’ end of the primer. When the blocker is an oligonucleotide, the blocker may comprise a sequence on its 5’ end that overlaps with a 3’ end sequence of a primer. Similarly, the blocker oligonucleotide may comprise: (i) at least one base present in the non-target nucleic acid but not present in the plurality of variants; (ii) one or more locked nucleic acids (LNAs); or (iii) a blocking moiety at its 3’ end to prevent extension by a DNA polymerase. For embodiments under (i), the at least one base may be present at the 5’ end or 3’ end of the blocker. In some embodiments, binding or hybridization of the blocker to the non-target nucleic acid (or an amplicon thereof) prevents a probe from annealing to the amplicon.

### Partitions / Amplification / Detection

Both methods described above include forming partitions from the sample, subjecting the partitions to conditions that promote nucleic acid amplification, and detecting a signal.

In some embodiments, the partitions of the plurality of partitions are aqueous droplets. For these embodiments, the aqueous droplets may be surrounded by an immiscible fluid (e.g., an oil such as a fluorinated oil or a silicone oil). Alternatively, in some embodiments, the partitions of the plurality of partitions are microfluidic wells.

Regarding amplification, the subjecting step may comprise thermocycling the partitions or methods of isothermal amplification. For those embodiments which utilize thermocycling, the nucleic acid amplification process may comprise a polymerase chain reaction (PCR).

The amplification reagents may comprise a polymerase having 5’ to 3’ exonuclease activity. For embodiments utilizing a mediator hydrolysis probe, cleavage of the 5’ end sequence may be mediated by a polymerase having 5’ to 3’ exonuclease activity.

For those embodiments which utilize aqueous droplets, the detecting step may comprise flowing the droplets past an optical detector unit. The detector unit may comprise an excitation source and an optical detector. In some embodiments, the aqueous droplets may be individually contained within a reaction tube, a chamber (e.g., a chamber of a microfluidic chip), a well of a plate, or a microwell of a microfluidic chip. For these embodiments, the detecting step may be performed by imaging the droplets. The imaging may be performed in a chamber (e.g., in a microfluidic chip), optionally by imaging the droplets disposed in a monolayer. For those embodiments which utilize microfluidic wells, the detecting step may comprise imaging at least one of the microfluidic wells.

### The Sample

Both methods are performed on an aqueous sample. In most embodiments, the aqueous sample comprises DNA or RNA. Accordingly, at least one of the plurality of variants may be present in DNA or RNA in the sample.

In some embodiments, the sample may be or may be derived from a cell free DNA sample, a circulating tumor DNA sample, a biopsy, a liquid biopsy, a blood sample, a bodily fluid, a plasma sample, a bone marrow aspiration, a urine sample, a stool sample, a formalin-fixed paraffin-embedded (FFPE) sample, a frozen sample, cerebrospinal fluid, or sputum.

The sample may comprise at least one synthetic or artificial nucleic acid. Accordingly, in some embodiments, at least one of the variants comprises a synthetic or artificial nucleic acid.

Some embodiments prepare the sample before partitioning. Preparing the sample may include isolating DNA or RNA from a sample to produce the aqueous sample, isolating circulating tumor DNA (ctDNA) or cell free DNA (cfDNA) from a sample to produce the aqueous sample, or isolating circulating tumor cells (CTCs) from a sample and purifying DNA or RNA from the CTCs to produce the aqueous sample. In some embodiments, the aqueous sample comprises RNA, and the method further comprises reverse transcribing the RNA into cDNA after the partitioning step.

### Variants

Both methods provide that the sample includes a non-target nucleic acid as well as may include a plurality of variants thereof.

In some embodiments, the non-target nucleic acid is an allele of a gene. Further, the non-target nucleic acid may be a wild type allele of a gene. Similarly, in some embodiments, the plurality of variants comprise a variant allele of a gene. For these embodiments, one or more variants of the plurality of variants may comprise a non-wild type variant allele of a gene. For some embodiments, the non-target nucleic acid is a first non-wild type variant allele of a gene and one or more variants of the plurality of variants comprise an allele of the gene different from the first non-wild type variant allele.

The plurality of variants may comprise an insertion, a deletion, a substitution, a duplication, an inversion, a translocation, a fusion, or a mutation resulting in an amino acid substitution.

In some exemplary embodiments, the gene is selected from the group consisting of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEB PA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (Cllorf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLDI, POLE, PPARG, PPP2R1A, PPP2R2A, PRDM1, PRKAR1A, PRKCI, PTCHI, PTEN, PTPN11, PTPRO, QKI, RAC1, RAD21, RAD51, RAD51B, RAD51C, RAD51D, RAD52, RAD54L, RAFI, RARA, RBI, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSCI, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTEA-4, dMMR, EGFR, ERBB1, ERBB2, FGFR1-3, FLT3, GD2, HDAC, HER1, HER2, HR, IDH2, IL-ip, IL-6, IL-6R, JAK1, JAK2, JAK3, KIT, KRAS, MEK, MET, MSI-H, mTOR, PARP, PD-1, PDGFR, PDGFRa, PDGFRp, PD-L1, PI3K5, PIGF, PTCH, RAF, RANKL, RET, R0S1, SLAMF7, VEGF, VEGFA, and VEGFB. In some embodiments, the gene is selected from EGFR, NRAS, BRAF, KRAS, ESR1 and PIK3CA.

In specific embodiments, the gene is EGFR. For these embodiments, the plurality of variants may comprise: V769_D770insASV; S768_D770dup; D770_N771insG; N771_H773dup; P772_H773dup; H773_V774insNPH; H773dup; E746_A750del; L747_P753delinsS; L747_E749del; L747_P753delinQ; L747_P750delinsP; L747_A750delinsP; an amino acid substitution at G719, optionally G719A, G719S, or G719C; an amino acid substitution at T790, optionally T790M; an amino acid substitution at C797, optionally C797S; a deletion or insertion in exon 18 of EGFR; a deletion or insertion in exon 19 of EGFR; a deletion or insertion in exon 20 of EGFR; an amino acid substitution at L861, optionally L861Q; or an amino acid substitution at L858, optionally L858R. Further, the plurality of variants may comprise: c.2303_2311dup (V769_D770insASV); c.2307_2308insGCCAGCGTG (S768_D770dup); c.2310_2311insGGT (D770_N771insG); c.2311_2319dup (N771_H773dup); c.2314_2319dup (P772_H773dup); c.2319-2320insAAACCCCAC (H773_V774insNPH); c.2317_2319dup (H773dup);c.2235_2249del (E746_A750del); c.2236_2250del (E746_A750del); c.2240_2257del (L747_P753delinsS); c.2239_2247del (L747_E749del); c.2239_2258delinsCA (L747_P753delinQ); c.2239_2248delinsC (L747_P750delinsP); c.2238_2248delinsGC (L747_A750delinsP); c.2156G>C (G719A); c.2155G>A (G719S); c.2155G>T (G719C); c.2369C>T (T790M); c.2390T>A (C797S-T>A); or c.2390G>C (C797S-G>C).

For additional embodiments, the gene is EGFR, and the plurality of variants may comprise one or more insertions such as: V769_D770insASV; S768_D770dup; D770_N771insG; N771_H773dup; P772_H773dup; H773_V774insNPH; or H773dup. Additional insertions may include: c.2303_2311dup (V769_D770insASV); c.2307_2308insGCCAGCGTG (S768-D770dup); c.2310_2311insGGT (D770_N771insG); c.2311_2319dup (N771_H773dup); c.2314_2319dup (P772_H773dup); c.2319-2320insAAACCCCAC (H773_V774insNPH); or c.2317_2319dup (H773dup).

For additional embodiments, the gene is EGFR, and the plurality of variants may comprise one or more deletions such as: E746_A750del; E746_A750del; L747_P753delinsS; L747_E749del; L747_P753delinQ; L747_P750delinsP; or L747_A750delinsP. Additional deletions may include: c.2235_2249del (E746_A750del); c.2236_2250del (E746_A750del); c.2240_2257del (L747_P753delinsS); c.2239_2247del (L747_E749del); c.2239_2258delinsCA (L747_P753delinQ); c.2239_2248delinsC (L747_P750delinsP); or c.2238_2248delinsGC (L747_A750delinsP).

For additional embodiments, the gene is EGFR, and the plurality of variants comprises one or more amino acid substitutions, such as: an amino acid substitution of G719, optionally G719A, G719S, or G719C; an amino acid substitution of T790, optionally T790M; or an amino acid substitution of C797, optionally C797S. Additional substitutions may include: c.2156G>C (G719A); c.2155G>A (G719S); c.2155G>T (G719C); c.2369C>T (T790M); c.2390T>A (C797S-T>A); or c.2390G>C (C797S-G>C).

In other embodiments, the gene is BRAF, and the plurality of variants may comprise a BRAF V600 amino acid substitution, optionally V600E. In yet other embodiments, the gene is KRAS, and the plurality of variants may comprise a KRAS G12 amino acid substitution, optionally G12C.

As identified above, the plurality of variants comprises one or more mutations resulting in an amino acid substitution. For example, when the non-target nucleic acid is an EGFR gene: (i) the first variant may be a mutation resulting in a G719 amino acid substitution, optionally G719A, G719S, or G719C, and the second variant may be a mutation resulting in a T790 amino acid substitution, optionally T790M; (ii) the first variant may be a mutation resulting in a G719 amino acid substitution, optionally G719A, G719S, or G719C, and the second variant may be a mutation resulting in a C797 amino acid substitution, optionally C797S; or (iii) the first variant may be a mutation resulting in a T790 amino acid substitution, optionally T790M, and the second variant may be a mutation resulting in a C797 amino acid substitution, optionally C797S.

In some embodiments, the mutation resulting in a G719A amino acid substitution is c.2156G>C; the mutation resulting in a G719S amino acid substitution is c.2155G>A; the mutation resulting in a G719C amino acid substitution is c.2155G>T; the mutation resulting in a T790M amino acid substitution is c.2369C>T; the mutation resulting in a C797S amino acid substitution is c.2390T>A; or the mutation resulting in a C797S amino acid substitution is c.2390G>C.

### Examples

### Example 1: Nondiscriminatory multiplexed digital PCR detection of nucleotide insertion mutations.

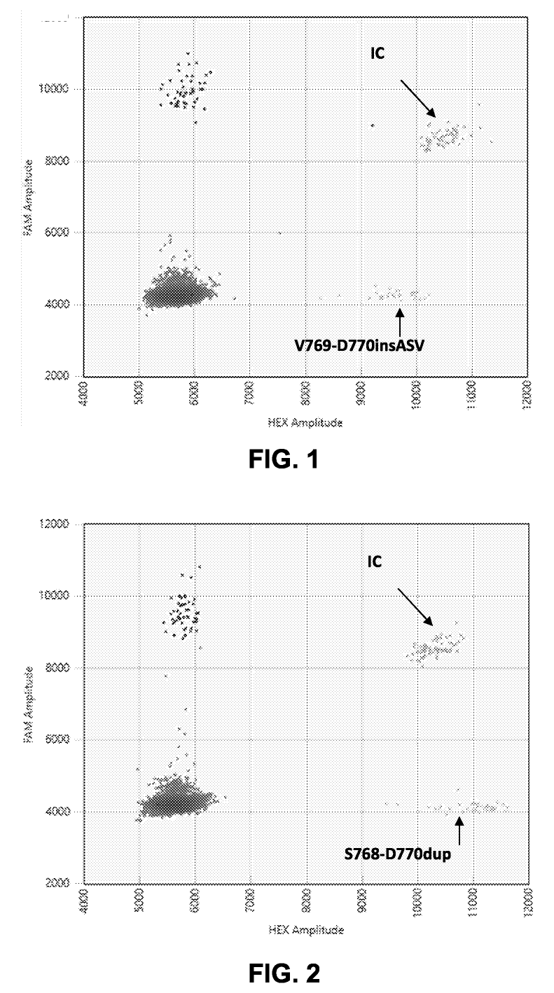

FIGS. 1-7 illustrate nondiscriminatory multiplexed digital PCR detection of seven insertions in the Epidermal Growth Factor Receptor (EGFR) gene combined with use of a wild type blocker to suppress signal from background wild type DNA. In this context, insertions are a type of genetic mutation where additional nucleotides are inserted into the DNA sequence of a gene.

Contrived samples were prepared containing 98.6% wild type EGFR background DNA and 1.4% DNA corresponding to one of the following seven EGFR exon 20 insertions:

c.2303_2311dup (V769_D770insASV)

c.2307_2308insGCCAGCGTG (S768_D770dup)

c.2310_2311insGGT (D770_N771insG)

c.2311_2319dup (N771_H773dup)

c.2314_2319dup (P772_H773dup)

c.2319-2320insAAACCCCAC (H773_V774insNPH)

c.2317_2319dup (H773dup)

Each sample also included digital PCR reagents including a primer pair for each of the insertions, a blocker oligonucleotide for the wild-type sequence, and a mixture of probes specific for each insertion and detectable in the HEX channel. All samples included an internal control (IC) detectable in both the FAM and HEX channels, and some samples contained additional gene sequences related to EGFR in the FAM and/or FAM/HEX channels.

The samples and reagents were partitioned into droplets using a droplet generator (Bio-Rad Automated Droplet Generator/ Bio-Rad QX200 Droplet Generator) followed by thermocycling. The droplets were then read on the Bio-Rad QX600 Droplet Reader. Each of the seven EGFR insertions was detected using probes detectable in the HEX channel.

FIGS. 1-7 provide results of digital PCR assays for contrived samples containing one of the seven EGFR insertions, as described above:

FIG. 1 shows the results for a sample containing c.2303_2311dup (V769_D770insASV),

FIG. 2 shows the results for a sample containing c.2307_2308insGCCAGCGTG (S768_D770dup),

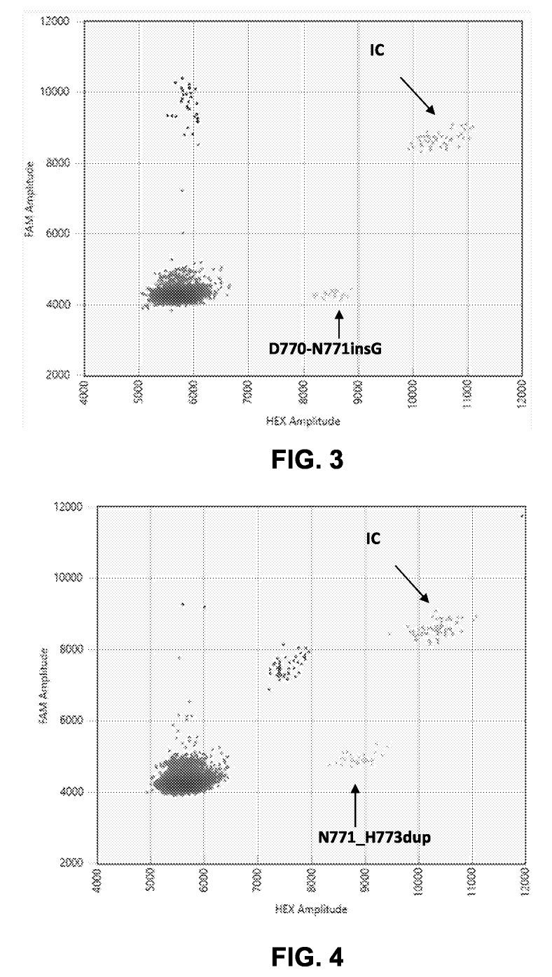

FIG. 3 shows the results for a sample containing c.2310_2311insGGT (D770_N771insG),

FIG. 4 shows the results for a sample containing c.2311_2319dup (N771_H773dup),

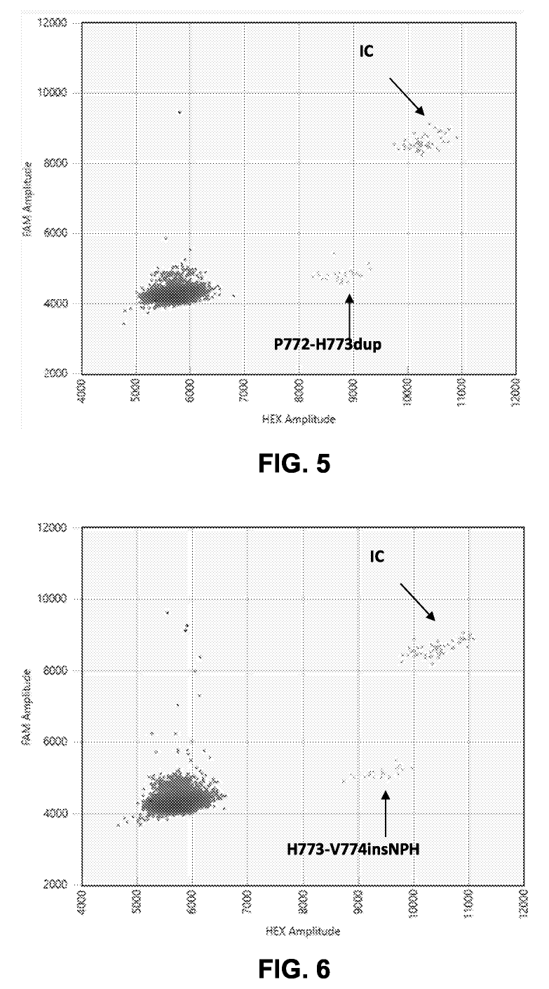

FIG. 5 shows the results for a sample containing c.2314_2319dup (P772_H773dup),

FIG. 6 shows the results for a sample containing c.2319-2320insAAACCCCAC (H773_V774insNPH), and

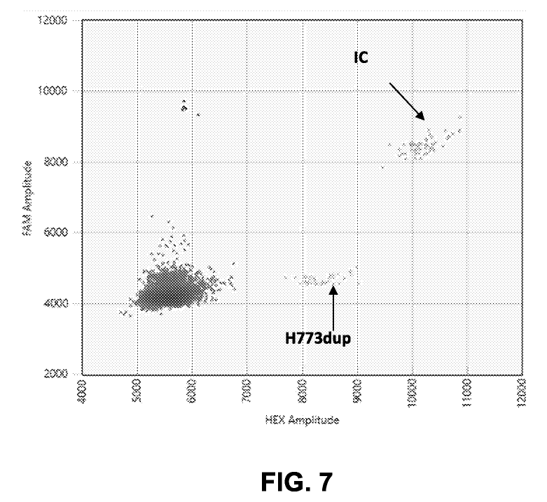

FIG. 7 shows the results for a sample containing c.2317_2319dup (H773dup).

In each of FIGS. 1-7, droplets with signal in the HEX channel are present, indicating the presence of at least one of the seven EGFR insertions.

The results in this Example demonstrate the use of a blocker oligonucleotide to inhibit signal from abundant background wild type DNA to enable successful detection of at least seven insertion mutations using highly multiplexed digital PCR in a non-discriminatory manner, since all of the mutations are detected in same detection channel (HEX).

Enabling detection of multiple mutations in a single cluster in one detection channel allows for expansion of the total number of variants that can be detected in a single assay. In this example, the EGFR exon 20 insertions that were chosen for non-discriminatory multiplexing are related and have the same clinical implication. Thus, discriminating between them does not impact the targeted therapy selection and/or patient management.

Further, in cases where samples are from liquid biopsy, the amount of cell free DNA in the samples is limited, with usual estimates being in the range of 5–10 ng per ml of plasma, which corresponds to 1,500 to 3,000 copies of the haploid human genome (Danesi et al., Clin Chim Acta. 2021 Sep:520:168-171). Furthermore, most of this DNA originates from normal cells, and circulating tumor DNA represents only a minor fraction, possibly as little as 0.1% of the total DNA, and even less in some cases. Thus, expanding the total number of variants detectable in a single digital PCR assay and inhibiting signal from wild type background using a blocker are beneficial.

### Example 2 : Nondiscriminatory multiplexed digital PCR detection of nucleotide deletion mutations.

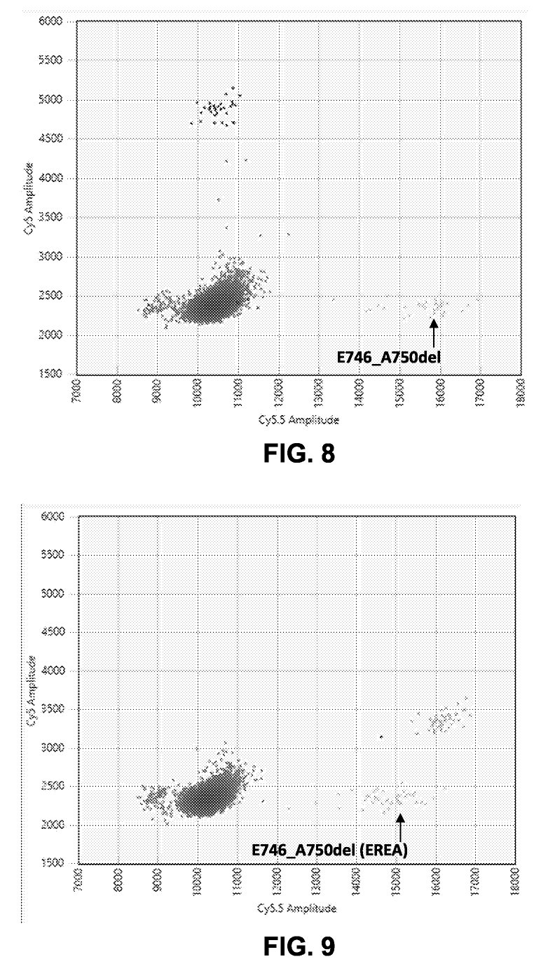

FIGS. 8-14 illustrate nondiscriminatory multiplexing of a digital PCR assay for seven deletions in the EGFR gene combined with use of a wild type blocker to suppress signal from background wild type DNA. In this context, deletions are a type of genetic mutation where one or more nucleotides are removed from the DNA sequence of a gene.

Contrived samples were prepared containing 98.6% wild type EGFR background DNA and 1.4% DNA corresponding to one of the following seven EGFR exon 19 deletions:

c.2235_2249del (E746_A750del)

c.2236_2250del (E746_A750del)

c.2240_2257del (L747_P753delinsS)

c.2239_2247del (L747_E749del)

c.2239_2258delinsCA (L747_P753delinQ)

c.2239_2248delinsC (L747_P750delinsP)

c.2238_2248delinsGC (L747_A750delinsP),

Each sample also included digital PCR reagents including a primer pair for each of the deletions, a blocker oligonucleotide for the wild-type sequence, and a mixture of probes specific for each deletion and detectable in the Cy5.5 channel, as well as a mixture of probes for other targets. All samples included an internal control (not shown in FIGS. 8-14), and some samples contained additional gene sequences related to EGFR or BRAF, which were detected in the Cy5 and Cy5/Cy5.5 channels. All of the samples also included a general probe specific to the wild-type sequence, targeting the non-deleted regions of the gene, enabling the detection of the seven deletions.

The samples and reagents were partitioned into droplets using a droplet generator (Bio-Rad Automated Droplet Generator/ Bio-Rad QX200 Droplet Generator) followed by thermocycling. The droplets were then read on the Bio-Rad QX600 Droplet Reader. Detection of all seven deletions was performed in the Cy5.5 channel.

FIGS. 8-14 provide results of digital PCR assays for contrived samples containing one of the seven EGFR deletions as described above:

FIG. 8 shows the results for a sample containing c.2235_2249del (E746_A750del),

FIG. 9 shows the results for a sample containing c.2236_2250del (E746_A750del),

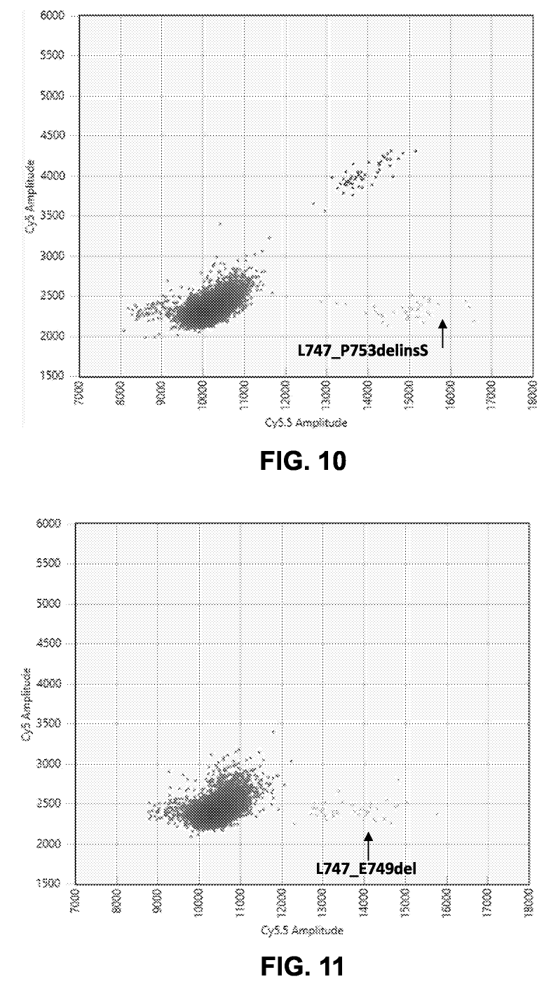

FIG. 10 shows the results for a sample containing c.2240_2257del (L747_P753delinsS),

FIG. 11 shows the results for a sample containing c.2239_2247del (L747_E749del),

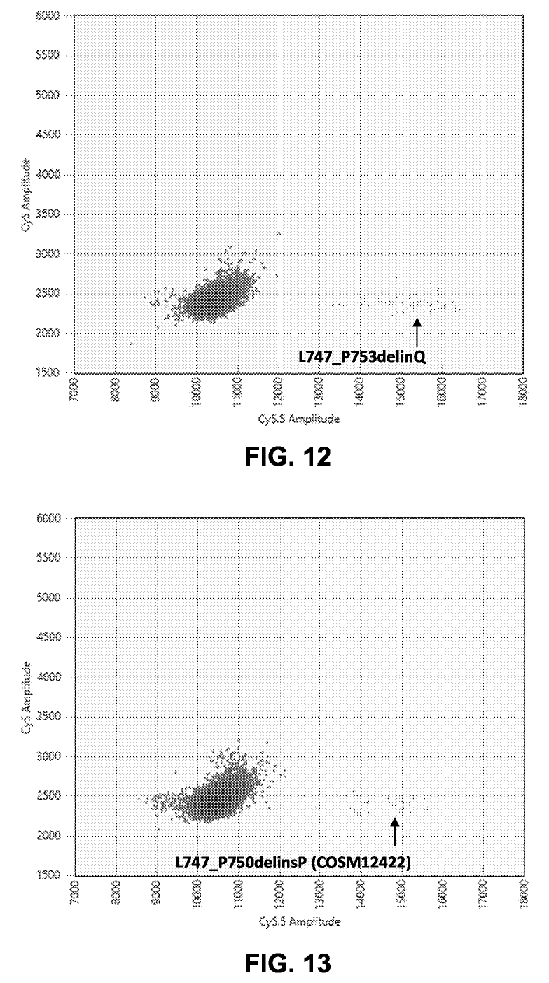

FIG. 12 shows the results for a sample containing c.2239_2258delinsCA (L747_P753delinQ),

FIG. 13 shows the results for a sample containing c.2239_2248delinsC (L747_P750delinsP),

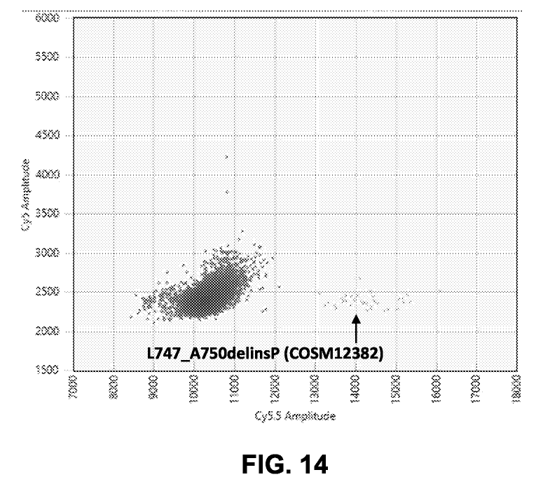

FIG. 14 shows the results for a sample containing c.2238_2248delinsGC (L747_A750delinsP).

In each of FIGS. 8-14, droplets with signal in the Cy5.5 channel are present, indicating the presence of at least one of the seven EGFR deletions. The assays can also detect presence of other deletions in exon 19 of the EGFR gene.

Similar to Example 1, the results in this Example demonstrate successful use of a blocker oligonucleotide to inhibit signal from abundant background wild type DNA to enable detection of at least seven deletion mutations using highly multiplexed digital PCR in a non-discriminatory manner (all deletions detected in the Cy5.5 channel). As discussed above, this strategy allows for the expansion of the number of total variants that can be assessed in a single multiplexed digital PCR assay, even in the presence of abundant wild type DNA.

### Example 3 : Nondiscriminatory multiplexed digital PCR detection of mutations resulting in amino acid substitutions.

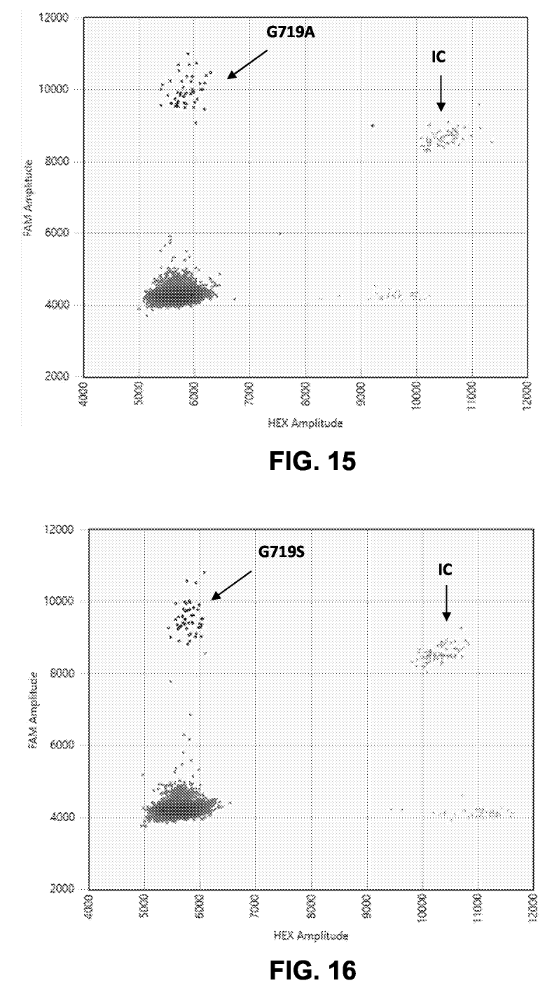

FIGS. 15-17 illustrate nondiscriminatory digital PCR multiplexing for three amino acid substitutions in the EGFR gene combined with use of a wild type blocker to suppress signal from background wild type DNA.

Contrived samples were prepared containing 98.6% wild type EGFR background DNA and 1.4% DNA corresponding to one of the following EGFR G719 amino acid substitutions in exon 18:

c.2156G>C (G719A)

c.2155G>A (G719S)

c.2155G>T (G719C)

Each sample also included digital PCR reagents including a primer pair for each of the G719 amino acid substitution variants, a blocker oligonucleotide for the wild-type sequence, and a mixture of probes specific for each variant and detectable in the FAM channel. All samples included an internal control (IC) detectable in both the FAM/HEX channels, and some samples contained additional gene sequences related to EGFR in the HEX and/or FAM/HEX channels.

The samples and reagents were partitioned into droplets using a droplet generator (Bio-Rad Automated Droplet Generator/ Bio-Rad QX200 Droplet Generator) followed by thermocycling. The droplets were then read on the Bio-Rad QX600 Droplet Reader. Each of the three EGFR mutations was detected using probes detectable in the FAM channel.

FIGS. 15-17 provide results of digital PCR assays for contrived samples containing one of the EGFR variants as described above:

FIG. 15 shows the results for a sample containing c.2156G>C (G719A),

FIG. 16 shows the results for a sample containing c.2155G>A (G719S), and

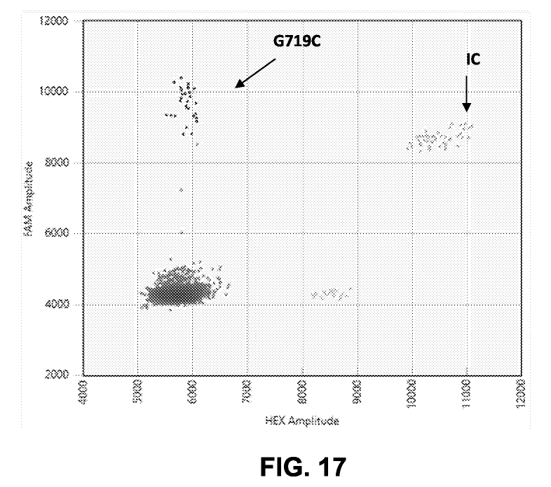

FIG. 17 shows the results for a sample containing c.2155G>T (G719C).

In each of FIGS. 15-17, droplets with signal in the FAM channel are present, indicating the presence of at least one of the EGFR G719 amino acid substitution variants.

Similar to Examples 1-2, the results in this Example demonstrate successful use of a blocker oligonucleotide to inhibit signal from abundant background wild type DNA to enable detection of amino acid substitution mutations using highly multiplexed digital PCR in a non-discriminatory manner (all mutations detected in the FAM channel).

### Example 4: Nondiscriminatory multiplexed digital PCR for assessment of cis/trans configurations of mutations.

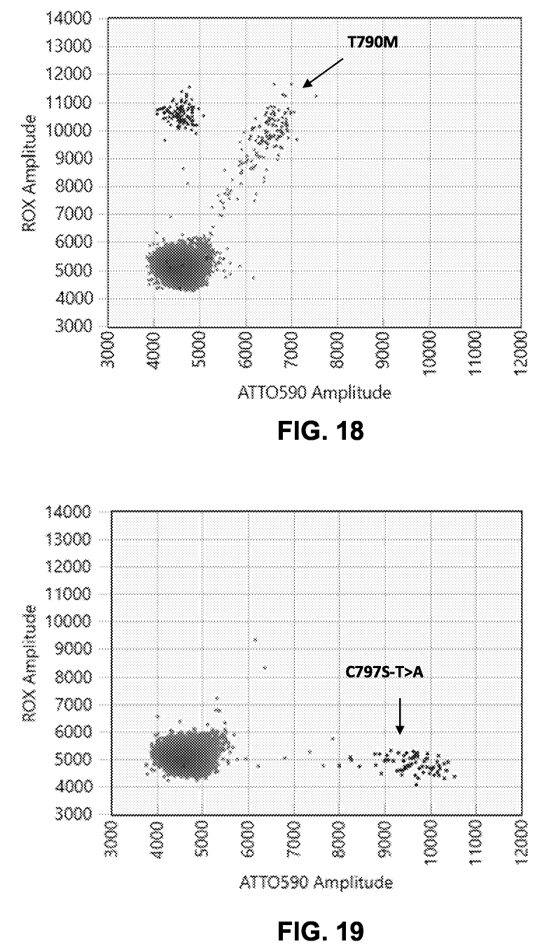

FIGS. 18-24 illustrate digital PCR multiplexing for variants in the EGFR gene to ascertain if two nearby amino acid changes (C797S and T790M) arise from mutations in the same or different alleles, which can be an important determination for understanding EGFR-based inhibitor resistance. This assay helps to determine whether the C797S mutations are present in cis (on the same allele) or trans (on a different allele) with the T790M mutation. Trans-configured mutations partition independently from each other into droplets, therefore co-partitioning is governed by Poisson distribution. Cis-configured mutations tend to co-segregate into the same droplets, because they are physically linked, and co-partitioning greatly exceeds expectation based on Poisson distribution. The C797S variant can arise from two nucleic acid changes, c.2390T>A (C797S-T>A) and c.2390G>C (C797S-G>C), so in total, three nucleic acid variants were assessed.

Contrived samples were prepared containing 98.6% wild type EGFR background DNA and 1.4% DNA corresponding to one of the following EGFR exon 20 variants:

c.2369C>T (T790M)

c.2390T>A (C797S-T>A)

c.2390G>C (C797S-G>C)

Each sample also included digital PCR reagents including a primer pair for each of the three variants, a blocker oligonucleotide for the wild-type sequence, and a mixture of probes specific for each variant and detectable in the ROX/ATTO590 or ATTO590 channels (the T790M mutation was detectable with probes in both the ROX and ATTO590 channels, while both C797S mutations were detectable with probes only in the ATTO590 channel in a nondiscriminatory manner). All samples included an internal control (not shown in FIGS. 18-24), and some samples contained additional gene sequences related to KRAS in the ROX channel.

The samples and reagents were partitioned into droplets using a droplet generator (Bio-Rad Automated Droplet Generator/ Bio-Rad QX200 Droplet Generator) followed by thermocycling. The droplets were then read on the Bio-Rad QX600 Droplet Reader.

FIGS. 18-24 provide results of digital PCR assays for contrived samples containing the EGFR variants as described above either alone or in combination in cis or trans:

FIG. 18 shows the results for a sample containing only T790M was present,

FIG. 19 shows the results for a sample containing only C797S-T>A was present,

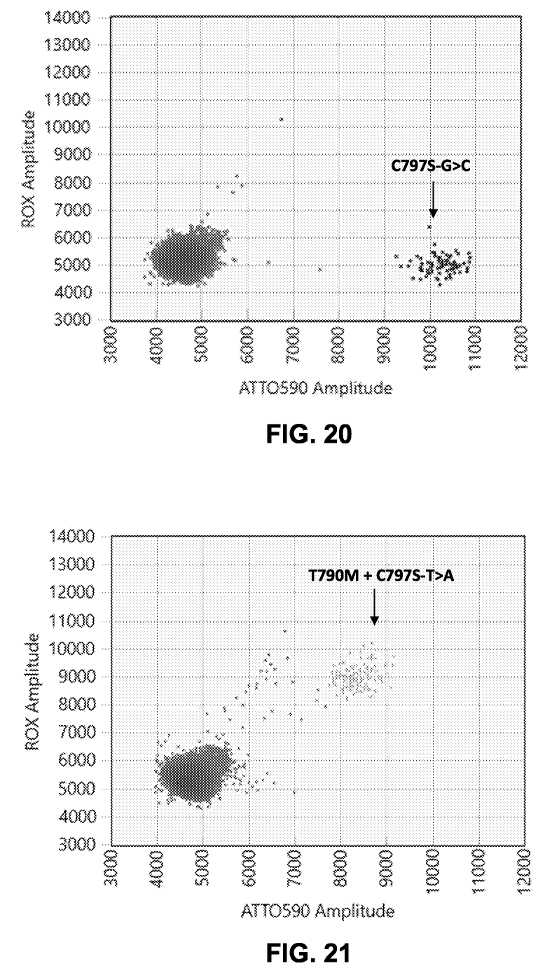

FIG. 20 shows the results for a sample containing only C797S-G>C was present,

FIG. 21 shows the results for a sample containing T790M and C797S-T>A were present in the same allele (cis),

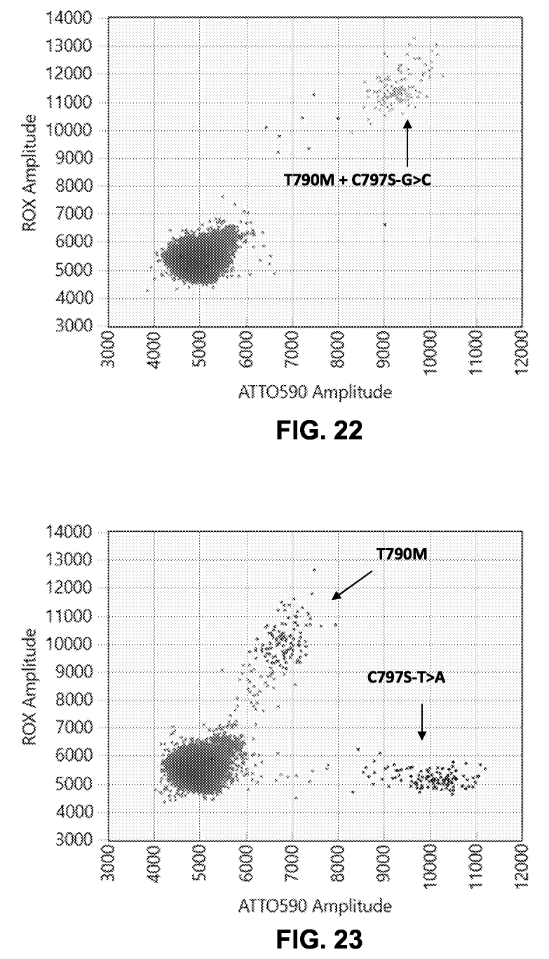

FIG. 22 shows the results for a sample containing T790M and C797S-G>C were present in the same allele (cis),

FIG. 23 shows the results for a sample containing T790M and C797S-T>A were present on different alleles (trans), and

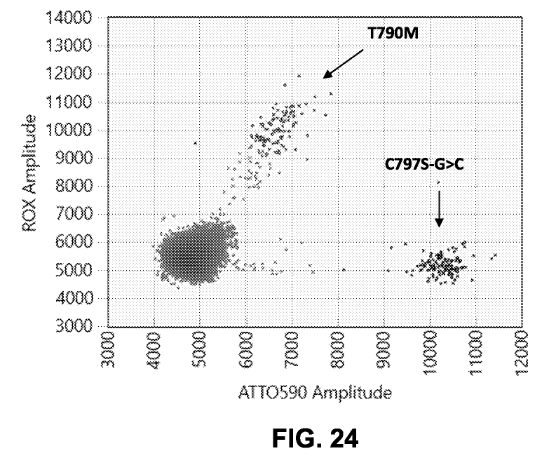

FIG. 24 shows the results for a sample containing T790M and C797S-G>C were present on different alleles (trans).

The results in this Example demonstrate the use of a blocker oligonucleotide to inhibit signal from abundant background wild type DNA to enable multiplexed detection of mutations in a non-discriminatory manner, as well as assessment of the cis/trans configuration of the mutations. As discussed above, this strategy allows for the expansion of the number of total variants that can be detected and assessed for cis/trans configuration in a single multiplexed digital PCR assay, even in the presence of abundant wild type DNA.

Features described above as well as those identified below may be combined in various combinations without departing from the scope of the invention. The following embodiments illustrate some possible, non-limiting combinations:

(Al) A detection method comprising: (a) partitioning into a plurality of partitions an aqueous sample that comprises a non-target nucleic acid and potentially comprises a plurality of variants of the non-target nucleic acid, wherein the partitions include: nucleic acid amplification reagents including a set of primers to amplify at least a portion of each of the plurality of variants, if present; a set of probes that detects at least two variants of the plurality of variants, and a blocker that inhibits amplification of the non-target nucleic acid; (b) subjecting the partitions to conditions that promote nucleic acid amplification, wherein the set of probes produces a first detectable signal upon amplification of one or more of the at least two variants; and (c) detecting the presence or absence of at least one variant of the plurality of variants in the sample based on detecting the first detectable signal from one or more of the partitions.

(A2) For the method denoted as (A1), the method further comprises detecting the presence of at least one variant of the plurality of variants in the sample based on detecting the first detectable signal from one or more of the partitions.

(A3) For the method denoted as (A1) or (A2), the method further comprises detecting the first detectable signal from one or more of the partitions indicates presence in the sample of at least one of the at least two variants.

(A4) For the method denoted as any one of (A1)-(A3), the detecting step does not discriminate between different variants of the plurality of variants.

(A5) For the method denoted as any one of (A1)-(A4), the detecting step does not determine which specific variant or variants are present in the sample.

(A6) For the method denoted as any one of (A1)-(A5), the method further comprises reporting the presence of at least one of the plurality of variants in the sample based on detection of the first detectable signal.

(A7) For the method denoted as (A6), the method further comprises reporting the presence of at least one variant of the plurality of variants in the sample without reporting which specific variant or variants of the plurality of variants are present in the sample.

(A8) For the method denoted as any one of (A1)-(A7), each probe of the set of probes comprises a detectable label that produces the first detectable signal upon amplification of its corresponding variant.

(A9) For the method denoted as any one of (A1)-(A8), at least two probes of the set of probes comprise the same detectable label that produces the first detectable signal.

(A10) For the method denoted as any one of (A1)-(A9), at least two different probes of the set of probes comprise different detectable labels that each produce the first detectable signal.

(A11) For the method denoted as any one of (A8)-(A10), the detectable label is an optically detectable label.

(A12) For the method denoted as any one of (A8)-(A11), the detectable label is a fluorescent label, a colorimetric label, or a chemiluminescent label; optionally wherein the fluorescent label is a fluorophore or a fluorescent protein.

(A13) For the method denoted as any one of (A1)-(A12), the first detectable signal is a fluorescent signal, a colorimetric signal, or a chemiluminescent signal.

(A14) For the method denoted as any one of (A1)-(A13), the first detectable signal is a fluorescent signal detectable in a first optical detection channel.

(A15) For the method denoted as any one of (A1)-(A12), the first detectable signal comprises a first fluorescent signal detectable in a first optical detection channel and a second fluorescent signal detectable in a second optical detection channel.

(A16) For the method denoted as any one of (A1)-(A7), the set of probes comprises a mediator hydrolysis probe comprising: a 3’ end sequence that hybridizes to a variant, and a cleavable 5’ end sequence that does not hybridize to the variant and that hybridizes to a universal reporter present in the partitions after cleavage occurs upon amplification of the variant, the universal reporter producing a detectable signal upon hybridizing to the cleaved 5’ end sequence of the mediator hydrolysis probe.

(A17) For the method denoted as (A16), the universal reporter comprises a detectable label that produces the detectable signal.

(A18) For the method denoted as (A17), the detectable label is an optically detectable label.

(A19) For the method denoted as (A17) or (A18), the detectable label is a fluorescent label, a colorimetric label, or a chemiluminescent label; optionally the fluorescent label is a fluorophore or a fluorescent protein.

(A20) For the method denoted as any one of (A16)-(A19), the set of probes are mediator hydrolysis probes.

(A21) For the method denoted as any one of (A16)-(A20), the set of probes comprises a first mediator hydrolysis probe specific to a first variant and a second mediator hydrolysis probe specific to a second variant, and the universal reporter produces the first detectable signal upon hybridizing to the cleaved 5’ end sequence of the first mediator hydrolysis probe and/or of the second mediator hydrolysis probe.

(A22) For the method denoted as (A21), the universal reporter comprises a detectable label that produces the first detectable signal.

(A23) For the method denoted as (A22), the detectable label is a fluorescent label, a colorimetric label, or a chemiluminescent label; optionally the fluorescent label is a fluorophore or a fluorescent protein.

(A24) For the method denoted as any one of (A20)-(A23), the first detectable signal is a fluorescent signal, a colorimetric signal, or a chemiluminescent signal.

(A25) For the method denoted as any one of (A20)-(A24), the first detectable signal is a fluorescent signal detectable in a first optical detection channel.

(A26) For the method denoted as any one of (A20)-(A24), the first detectable signal comprises a first fluorescent signal detectable in a first optical detection channel and a second fluorescent signal detectable in a second optical detection channel.

(A27) For the method denoted as any one of (A1)-(A26), the set of probes comprises 1-25 or more probes.

(A28) For the method denoted as any one of (A1)-(A27), each probe of the set of probes produces the first detectable signal upon amplification of its corresponding variant.

(A29) For the method denoted as any one of (A1)-(A28), each probe of the set of probes is specific to a different variant of the plurality of variants, and each probe produces the first detectable signal upon amplification of its corresponding variant.

(A30) For the method denoted as any one of (A1)-(A29), the set of probes comprises 1-25 or more different probes, each probe is specific for a different variant of the plurality of variants, and each probe produces the first detectable signal upon amplification of its corresponding variant.

(A31) For the method denoted as any one of (A1)-(A30), the set of probes comprises one probe that is specific for 2-25, or more, different variants of the plurality of variants, and said one probe produces the first detectable signal upon amplification of one or more of said different variants.

(B1) A detection method comprising: (a) partitioning into a plurality of partitions an aqueous sample that comprises a non-target nucleic acid and potentially comprises a plurality of variants of the non-target nucleic acid, the partitions including: nucleic acid amplification reagents including a set of primers to amplify at least a portion of each of the plurality of variants; a set of probes that detect at least two variants of the plurality of variants, and a blocker that inhibits amplification of the non-target nucleic acid; (b) subjecting the partitions to conditions that promote nucleic acid amplification, the set of probes comprising: at least a first probe that is specific for a first variant of the plurality of variants and produces a first detectable signal upon amplification of the first variant, and at least a second probe that is specific for a second variant of the plurality of variants and produces a second detectable signal upon amplification of the second variant; and (c) detecting a cis or trans configuration of at least the first variant and the second variant in the sample by detecting the presence or absence of the first detectable signal and the second detectable signal in one or more partitions of the plurality of partitions, wherein: (i) presence of both the first detectable signal and the second detectable signal in one or more partitions of the plurality of partitions indicates that the first variant and the second variant are present in the sample in a cis configuration, and (ii) presence of only the first detectable signal in a first subset of partitions of the plurality of partitions and presence of only the second detectable signal in a second subset of partitions of the plurality of partitions indicates that the first variant and the second variant are present in the sample in a trans configuration.

(B2) For the method denoted as (B1), the first probe comprises a first detectable label that produces the first detectable signal upon amplification of the first variant and the second probe comprises a second detectable label that produces the second detectable signal upon amplification of the second variant.

(B3) For the method denoted as (B2), the first and second detectable labels are optically detectable labels.

(B4) For the method denoted as (B3), the optically detectable label is a fluorescent label, a colorimetric label, or a chemiluminescent label; optionally wherein the fluorescent label is a fluorophore or a fluorescent protein.

(B5) For the method denoted as any one of (B1)-(B4), the first detectable signal is a first fluorescent signal and the second detectable signal is a second fluorescent signal.

(B6) For the method denoted as any one of (B1)-(B5), (a) the first detectable signal is a first fluorescent signal detectable in a first optical detection channel and the second detectable signal is a second fluorescent signal detectable in a second optical detection channel; or (b) the first detectable signal is a first fluorescent signal at a first intensity detectable in a first optical detection channel and the second detectable signal is the first fluorescent signal at a second intensity different from the first intensity detectable in the first optical detection channel.

(B7) For the method denoted as any one of (B1)-(B4), (a) the first detectable signal comprises a first fluorescent signal detectable in a first optical detection channel and a second fluorescent signal detectable in a second optical detection channel; and/or (b) the second detectable signal comprises a third fluorescent signal detectable in a third optical detection channel and a fourth fluorescent signal detectable in a fourth optical detection channel.

(B8) For the method denoted as (B1), the set of probes comprises one or more mediator hydrolysis probes comprising: a 3’ end sequence that hybridizes to a variant, and a cleavable 5’ end sequence that does not hybridize to the variant and that hybridizes to a universal reporter present in the partitions after cleavage occurs upon amplification of the variant, the universal reporter producing a detectable signal upon hybridizing to the cleaved 5’ end sequence of the mediator hydrolysis probe.

(B9) For the method denoted as (B8), the universal reporter comprises a detectable label that produces the detectable signal upon hybridizing to the cleaved 5’ end sequence of a mediator hydrolysis probe.

(B10) For the method denoted as (B9), the detectable label is an optically detectable label.

(B11) For the method denoted as (B9) or (B10), the detectable label is a fluorescent label, a colorimetric label, or a chemiluminescent label; optionally wherein the fluorescent label is a fluorophore or a fluorescent protein.

(B12) For the method denoted as any one of claims (B8)-(B11), the first probe is a first mediator hydrolysis probe specific to the first variant and the second probe is a second mediator hydrolysis probe specific to the second variant, and (i) a first universal reporter present in the partitions produces the first detectable signal upon hybridizing to the cleaved 5’ end sequence of the first mediator hydrolysis probe; and/or (ii) a second universal reporter present in the partitions produces the second detectable signal upon hybridizing to the cleaved 5’ end sequence of the second mediator hydrolysis probe.

(B13) For the method denoted as (B12), the first universal reporter comprises a first detectable label that produces the first detectable signal upon hybridizing to the cleaved 5’ end sequence of the first mediator hydrolysis probe; and/or the second universal reporter comprises a second detectable label that produces the second detectable signal upon hybridizing to the cleaved 5’ end sequence of the second mediator hydrolysis probe.

(B14) For the method denoted as (B13), the first and second detectable labels are fluorescent labels, colorimetric labels, or chemiluminescent labels; optionally the fluorescent label is a fluorophore or a fluorescent protein.

(B15) For the method denoted as any one of (B8)-(B14), the first and second detectable signals are fluorescent signals.

(B16) For the method denoted as (B15), (a) the first detectable signal is a first fluorescent signal detectable in a first optical detection channel and the second detectable signal is a second fluorescent signal detectable in a second optical detection channel; or (b) the first detectable signal is a first fluorescent signal at a first intensity detectable in a first optical detection channel and the second detectable signal is the first fluorescent signal at a second intensity different from the first intensity detectable in the first optical detection channel.

(B17) For the method denoted as (B15), (a) the first detectable signal comprises a first fluorescent signal detectable in a first optical detection channel and a second fluorescent signal detectable in a second optical detection channel; and/or (b) the second detectable signal comprises a third fluorescent signal detectable in a third optical detection channel and a fourth fluorescent signal detectable in a fourth optical detection channel.

(B18) For the method denoted as any one of claims (B1)-(B17), the set of probes comprises 2-25, or more, probes.

(B19) For the method denoted as any one of (B1)-(B18), each probe of the set of probes produces either the first detectable signal or the second detectable signal upon amplification of its corresponding variant.

(B20) For the method denoted as any one of (B1)-(B19), each probe of the set of probes is specific to a different variant of the plurality of variants, and wherein each probe produces either the first detectable signal or the second detectable signal upon amplification of its corresponding variant.

(B21) For the method denoted as any one of (B1)-(B20), the set of probes comprises 2-25, or more, different probes, each probe is specific to a different variant of the plurality of variants, and each probe produces either the first detectable signal or the second detectable signal upon amplification of its corresponding variant.

(B22) For the method denoted as any one of (B1)-(B21), the first probe is specific to 1-25, or 2-25, or more, different variants of the plurality of variants, and said first probe produces the first detectable signal upon amplification of one or more of said different variants.

(B23) For the method denoted as any one of (B1)-(B22), the second probe is specific to 1-25, or 2-25, or more, different variants of the plurality of variants, and said second probe produces the second detectable signal upon amplification of one or more of said different variants.

(B24) For the method denoted as any one of (B1)-(B23), the set of probes comprises at least a third probe that is specific to a third variant and produces the first detectable signal upon amplification of the third variant; and/or the set of probes comprises at least a fourth probe that is specific to a fourth variant and produces the second detectable signal upon amplification of the fourth variant.

(B25) For the method denoted as any one of (B1)-(B24), the set of probes comprises 1-25, or 2-25, or more, different probes specific to a different variant of the plurality of variants that each produce the first detectable signal upon amplification of its corresponding variant; and/or the set of probes comprises 1-25, or 2-25, or more, different probes specific to a different variant of the plurality of variants that each produce the second detectable signal upon amplification of its corresponding variant.

(C1) For the method denoted as any one of (A1)-(A31) or (B1)-(B25), the blocker does not comprise a detectable label.

(C2) For the method denoted as any one of (A1)-(A31), (B1)-(B25), or (C1), the blocker comprises an oligonucleotide, a protein, or an RNA-guided binding protein.

(C3) For the method denoted as any one of (A1)-(A31), (B1)-(B25), or (C1)-(C2), the blocker blocks binding of a primer to the non-target nucleic acid.

(C4) For the method denoted as any one of (A1)-(A31), (B1)-(B25), or (C1)-(C3), the blocker does not block binding of a primer to one or more variants of the plurality of variants.

(C5) For the method denoted as (C3), the blocker blocks binding of the 3’ end of the primer.

(C6) For the method denoted as (C5), the blocker blocks binding of the 3’ end of the primer to the non-target nucleic acid.

(C7) For the method denoted as any one of (A1)-(A31), (B1)-(B25), or (C1)-(C6), the blocker is an oligonucleotide.

(C8) For the method denoted as (C7), the blocker comprises a sequence on its 5’ end that overlaps with a 3’ end sequence of a primer.

(C9) For the method denoted as (C7) or (C8), the blocker oligonucleotide comprises one or more of: (i) at least one base present in the non-target nucleic acid but not present in the plurality of variants, optionally wherein said at least one base is present at the 5’ end or 3’ end of the blocker; (ii) one or more locked nucleic acids (LNAs); or (iii) a blocking moiety at its 3’ end to prevent extension by a DNA polymerase.

(C10) For the method denoted as any one of (A1)-(A31), (B1)-(B25), or (C1)-(C9), binding or hybridization of the blocker to the non-target nucleic acid prevents a primer from annealing to the non-target nucleic acid.

(C11) For the method denoted as any one of (A1)-(A31), (B1)-(B25), or (C1)-(C10), binding or hybridization of the blocker to an amplicon of the non-target nucleic acid prevents a probe from annealing to the amplicon.

(C12) For the method denoted as any one of (A1)-(A31), (B1)-(B25), or (C1)-(C11), the subjecting step comprises thermocycling the partitions.

(C13) For the method denoted as any one of (A1)-(A31), (B1)-(B25), or (C1)-(C12), the nucleic acid amplification comprises polymerase chain reaction (PCR).

(C14) For the method denoted as any one of (A1)-(A31), (B1)-(B25), or (C1)-(C12), the nucleic acid amplification comprises isothermal amplification.

(C15) For the method denoted as any one of (A1)-(A31), (B1)-(B25), or (C1)-(C14), the partitions of the plurality of partitions are aqueous droplets.

(C16) For the method denoted as (C15), the aqueous droplets are surrounded by an immiscible fluid.

(C17) For the method denoted as (C16), the immiscible fluid is an oil, optionally a fluorinated oil or a silicone oil.

(C18) For the method denoted as any one of (A1)-(A31), (B1)-(B25), or (C1)-(C14), the partitions of the plurality of partitions are microfluidic wells.

(C19) For the method denoted as any one of (C15)-(C17), the detecting step comprises flowing the droplets past an optical detection unit, and optionally comprises an excitation source and an optical detector.

(C20) For the method denoted as any one of (C15)-(C17) or (C19), the aqueous droplets are individually contained within a reaction tube or well of a plate.

(C21) For the method denoted as any one of (C15)-(C17) or (C19)-(C20), the detecting step is performed by imaging the droplets.

(C22) For the method denoted as (C21), imaging is performed by imaging the droplets disposed in a chamber.

(C23) For the method denoted as (C21) or (C22), the detecting step comprises imaging the droplets disposed in a monolayer.

(C24) For the method denoted as (C18), the detecting step comprises imaging at least one microfluidic well.

(C25) For the method denoted as any one of (A1)-(A31), (B1)-(B25), or (C1)-(C24), at least one of the plurality of variants is present in DNA or RNA in the sample.

(C26) For the method denoted as any one of (A1)-(A31), (B1)-(B25), or (C1)-(C25), the aqueous sample comprises DNA or RNA.

(C27) For the method denoted as any one of (A1)-(A31), (B1)-(B25), or (C1)-(C26), the sample is or is derived from a cell free DNA sample, a circulating tumor DNA sample, a biopsy, a liquid biopsy, a blood sample, a bodily fluid, a plasma sample, a bone marrow aspiration, a urine sample, a stool sample, a formalin-fixed paraffin-embedded (FFPE) sample, a frozen sample, cerebrospinal fluid, or sputum.

(C28) For the method denoted as any one of (A1)-(A31), (B1)-(B25), or (C1)-(C27), the sample comprises at least one synthetic or artificial nucleic acid.

(C29) For the method denoted as (C28), at least one of the variants comprises a synthetic or artificial nucleic acid.

(C30) For the method denoted as any one of (A1)-(A31), (B1)-(B25), or (C1)-(C29), the method further comprises: isolating DNA or RNA from a sample to produce the aqueous sample; isolating circulating tumor DNA (ctDNA) or cell free DNA (cfDNA) from a sample to produce the aqueous sample; or isolating circulating tumor cells (CTCs) from a sample and purifying DNA or RNA from the CTCs to produce the aqueous sample.

(C31) For the method denoted as any one of (A1)-(A31), (B1)-(B25), or (C1)-(C30), the aqueous sample comprises RNA, and the method further comprises reverse transcribing the RNA into cDNA after the partitioning step.

(C32) For the method denoted as any one of (A1)-(A31), (B1)-(B25), or (C1)-(C31), the non-target nucleic acid is an allele of a gene.

(C33) For the method denoted as any one of (A1)-(A31), (B1)-(B25), or (C1)-(C32), the non-target nucleic acid is a wild type allele of a gene.

(C34) For the method denoted as any one of (A1)-(A31), (B1)-(B25), or (C1)-(C33), the plurality of variants comprises a variant allele of a gene.

(C35) For the method denoted as any one of (A1)-(A31), (B1)-(B25), or (C1)-(C34), one or more variants of the plurality of variants comprise a non-wild type variant allele of a gene.

(C36) For the method denoted as any one of (A1)-(A31), (B1)-(B25), or (C1)-(C35), the non-target nucleic acid is a wild type allele of a gene and one or more variants of the plurality of variants comprise a non-wild type variant allele of the gene.

(C37) For the method denoted as any one of (A1)-(A31), (B1)-(B25), or (C1)-(C35), the non-target nucleic acid is a first non-wild type variant allele of a gene and one or more variants of the plurality of variants comprise an allele of the gene different from the first non-wild type variant allele.

(C38) For the method denoted as any one of (A1)-(A31), (B1)-(B25), or (C1)-(C37), the plurality of variants comprises an insertion, a deletion, a substitution, a duplication, an inversion, a translocation, a fusion, or a mutation resulting in an amino acid substitution, or any combination thereof.

(C39) For the method denoted as any one of (C32)-(C38), the gene is selected from the group consisting of EGFR, NRAS, BRAF, KRAS, ESR1, and PIK3CA.

(C40) For the method denoted as (C39), the gene is EGFR.

(C41) For the method denoted as (C40), the plurality of variants comprises: V769_D770insASV; S768_D770dup; D770_N771insG; N771_H773dup; P772_H773dup; H773_V774insNPH; H773dup; E746_A750del; L747_P753delinsS; L747_E749del; L747_P753delinQ; L747_P750delinsP; L747_A750delinsP; an amino acid substitution at G719, optionally G719A, G719S, or G719C; an amino acid substitution at T790, optionally T790M; an amino acid substitution at C797, optionally C797S; a deletion or insertion in exon 18 of EGFR; a deletion or insertion in exon 19 of EGFR; a deletion or insertion in exon 20 of EGFR; an amino acid substitution at L861, optionally L861Q; or an amino acid substitution at L858, optionally L858R.

(C42) For the method denoted as (C40) or (C41), the plurality of variants comprises: c.2303_2311dup (V769_D770insASV); c.2307_2308insGCCAGCGTG (S768_D770dup); c.2310_2311insGGT (D770_N771insG); c.2311_2319dup (N771_H773dup); c.2314_2319dup (P772_H773dup); c.2319-2320insAAACCCCAC (H773_V774insNPH); c.2317_2319dup (H773dup); c.2235_2249del (E746_A750del); c.2236_2250del (E746_A750del); c.2240_2257del (L747_P753delinsS); c.2239_2247del (L747_E749del); c.2239_2258delinsCA (L747_P753delinQ); c.2239_2248delinsC (L747_P750delinsP); c.2238_2248delinsGC (L747_A750delinsP); c.2156G>C (G719A); c.2155G>A (G719S); c.2155G>T (G719C); c.2369C>T (T790M); c.2390T>A (C797S-T>A); or c.2390G>C (C797S-G>C).

(C43) For the method denoted as (C39), the gene is BRAF, and optionally the plurality of variants comprises a BRAF V600 amino acid substitution, optionally V600E.

(C44) For the method denoted as (C39), the gene is KRAS, and optionally the plurality of variants comprises a KRAS G12 amino acid substitution, optionally G12C.

(C45) For the method denoted as (C39), the gene is EGFR, and the plurality of variants comprises one or more insertions, and optionally the plurality of variants comprises: V769_D770insASV; S768_D770dup; D770_N771insG; N771_H773dup; P772_H773dup; H773_V774insNPH; or H773dup.

(C46) For the method denoted as (C45), the plurality of variants comprises: c.2303_2311dup (V769_D770insASV); c.2307_2308insGCCAGCGTG (S768_D770dup); c.2310_2311insGGT (D770_N771insG); c.2311_2319dup (N771_H773dup); c.2314_2319dup (P772_H773dup); c.2319-2320insAAACCCCAC (H773_V774insNPH); or c.2317_2319dup (H773dup).

(C47) For the method denoted as (C39), the gene is EGFR, and the plurality of variants comprises one or more deletions, and optionally the plurality of variants comprises: E746_A750del; E746_A750del; L747_P753delinsS; L747_E749del; L747_P753delinQ; L747_P750delinsP; or L747_A750delinsP.

(C48) For the method denoted as (C47), the plurality of variants comprises: c.2235_2249del (E746_A750del); c.2236_2250del (E746_A750del); c.2240_2257del (L747_P753delinsS); c.2239_2247del (L747_E749del); c.2239_2258delinsCA (L747_P753delinQ); c.2239_2248delinsC (L747_P750delinsP); or c.2238_2248delinsGC (L747_A750delinsP).

(C49) For the method denoted as (C39), the gene is EGFR, the plurality of variants comprises one or more amino acid substitutions, and optionally the plurality of variants comprises: an amino acid substitution of G719, optionally G719A, G719S, or G719C; an amino acid substitution of T790, optionally T790M; or an amino acid substitution of C797, optionally C797S.

(C50) For the method denoted as (C49), the plurality of variants comprises: c.2156G>C (G719A); c.2155G>A (G719S); c.2155G>T (G719C); c.2369C>T (T790M); c.2390T>A (C797S-T>A); or c.2390G>C (C797S-G>C).

(C51) For the method denoted as any one of (B1)-(B25) or (C1)-(C39), the plurality of variants comprises one or more mutations resulting in an amino acid substitution.

(C52) For the method denoted as (C51), the non-target nucleic acid is an EGFR gene, and optionally: (i) the first variant is a mutation resulting in a G719 amino acid substitution, optionally G719A, G719S, or G719C, and the second variant is a mutation resulting in a T790 amino acid substitution, optionally T790M; (ii) the first variant is a mutation resulting in a G719 amino acid substitution, optionally G719A, G719S, or G719C, and the second variant is a mutation resulting in a C797 amino acid substitution, optionally C797S; or (iii) the first variant is a mutation resulting in a T790 amino acid substitution, optionally T790M, and the second variant is a mutation resulting in a C797 amino acid substitution, optionally C797S.

(C53) For the method denoted as (C52), the mutation resulting in a G719A amino acid substitution is c.2156G>C; the mutation resulting in a G719S amino acid substitution is c.2155G>A; the mutation resulting in a G719C amino acid substitution is c.2155G>T; the mutation resulting in a T790M amino acid substitution is c.2369C>T; the mutation resulting in a C797S amino acid substitution is c.2390T>A; or the mutation resulting in a C797S amino acid substitution is c.2390G>C.

(C54) For the method denoted as any one of (A1)-(A31), (B1)-(B25), or (C1)-(C53), each probe of the set of probes comprises a detectable label that produces a detectable signal upon amplification of its corresponding variant.

(C55) For the method denoted as (C54), the detectable label is an optically detectable label.

(C56) For the method denoted as (C54) or (C55), the detectable label is a fluorescent label, a colorimetric label, or a chemiluminescent label; optionally the fluorescent label is a fluorophore or a fluorescent protein.

(C57) For the method denoted as any one of (A12)-(A15), (A19)-(A31), (B4)-(B7), (B11)-(B25), or (C1)-(C56), the fluorescent label is selected from carboxyfluorescein (FAM), hexachlorofluorescein (HEX), cyanine5 (CY5), Quasar 670, cyanine5.5 (CY5.5), Quasar 705, TYE 705, 5-carboxy-X-rhodamine (5-ROX), Cal Fluor Red 590, TYE665, a fluorescent oligonucleotide dye with 594 nm absorption, and ATTO590.

(C58) For the method denoted as any one of (A1)-(A31), (B1)-(B25), or (C1)-(C57), probes of the set of probes comprise molecular beacon probes.

(C59) For the method denoted as any one of (A1)-(A31), (B1)-(B25), or (C1)-(C58), probes of set of probes comprise fluorescent hydrolysis probes.

(C60) For the method denoted as any one of (A1)-(A31), (B1)-(B25), or (C1)-(C59), the amplification reagents comprise a polymerase having 5’ to 3’ exonuclease activity.

(C61) For the method denoted as any one of (A16)-(A31), (B8)-(B25), or (C1)-(C60), cleavage and release of the 5’ end sequence is mediated by a polymerase having 5’ to 3’ exonuclease activity.

(C62) For the method denoted as any one of (A1)-(A31), (C1)-(C50), or (C57)-(C61), the detecting step does not discriminate between the at least two variants.

(C63) For the method denoted as any one of (A1)-(A31), (C1)-(C50), or (C57)-(C62), the detecting step does not determine which specific variant or variants of the at least two variants is present in the sample.

(C64) For the method denoted as any one of (A1)-(A31), (C1)-(C50), or (C57)-(C63), the method further comprises reporting the presence of at least one of the at least two variants in the sample based on detection of the first detectable signal.

(C65) For the method denoted as (C64), the method further comprises reporting the presence of at least one of the at least two variants in the sample without reporting which specific variant or variants of the at least two variants is present in the sample.

(C66) For the method denoted as any one of (A1)-(A31), (C1)-(C50), or (C57)-(C65), the set of probes comprises one probe that detects the at least two variants and produces the first detectable signal upon amplification of one or more of the at least two variants.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A detection method comprising:
(a) partitioning an aqueous sample comprising a non-target nucleic acid and potentially comprising a plurality of variants of the non-target nucleic acid into a plurality of partitions, the partitions including:
a set of probes that detects at least two variants of the plurality of variants; and
a blocker that inhibits amplification of the non-target nucleic acid;
(b) subjecting the partitions to conditions that promote nucleic acid amplification, wherein the set of probes produces a first detectable signal upon amplification of one or more of the at least two variants; and
(c) detecting the presence or absence of at least one variant of the plurality of variants in the sample based on detecting the first detectable signal from one or more of the partitions.

2. The method of claim 1, wherein the presence of at least one variant of the plurality of variants is detected in the sample.

3. The method of claim 1, wherein the detecting step does not discriminate between different variants of the plurality of variants.

4. The method of claim 1, further comprising reporting the presence of at least one of the plurality of variants in the sample based on detection of the first detectable signal without reporting which specific variant or variants of the plurality of variants are present in the sample.

5. The method of claim 1, wherein at least two probes of the set of probes comprise the same detectable label that produces the first detectable signal.

6. The method of claim 1, wherein at least two different probes of the set of probes comprise different detectable labels that each produce the first detectable signal.

7. The method of claim 1, wherein the first detectable signal comprises a first fluorescent signal detectable in a first optical detection channel and a second fluorescent signal detectable in a second optical detection channel.

8. The method of claim 1, wherein each probe of the set of probes produces the first detectable signal upon amplification of its corresponding variant.

9. The method of claim 1, wherein each probe of the set of probes is specific to a different variant of the plurality of variants, and wherein each probe produces the first detectable signal upon amplification of its corresponding variant.

10. A detection method comprising:
(a) partitioning into a plurality of partitions an aqueous sample that comprises a non-target nucleic acid and potentially comprises a plurality of variants of the non-target nucleic acid, wherein the partitions include:
nucleic acid amplification reagents including a set of primers to amplify at least a portion of each of the plurality of variants;
a set of probes that detect at least two variants of the plurality of variants, and
a blocker that inhibits amplification of the non-target nucleic acid;
(b) subjecting the partitions to conditions that promote nucleic acid amplification, wherein the set of probes comprises:
at least a first probe that is specific for a first variant of the plurality of variants and produces a first detectable signal upon amplification of the first variant, and
at least a second probe that is specific for a second variant of the plurality of variants and produces a second detectable signal upon amplification of the second variant; and
(c) detecting a cis or trans configuration of at least the first variant and the second variant in the sample by detecting the presence or absence of the first detectable signal and the second detectable signal in one or more partitions of the plurality of partitions,
wherein: (i) presence of both the first detectable signal and the second detectable signal in one or more partitions of the plurality of partitions indicates that the first variant and the second variant are present in the sample in a cis configuration, and (ii) presence of only the first detectable signal in a first subset of partitions of the plurality of partitions and presence of only the second detectable signal in a second subset of partitions of the plurality of partitions indicates that the first variant and the second variant are present in the sample in a trans configuration.

11. The method of claim 10, wherein the first probe comprises a first detectable label that produces the first detectable signal upon amplification of the first variant and the second probe comprises a second detectable label that produces the second detectable signal upon amplification of the second variant.

12. The method of claim 10, wherein (a) the first detectable signal is a first fluorescent signal detectable in a first optical detection channel and the second detectable signal is a second fluorescent signal detectable in a second optical detection channel; or (b) the first detectable signal is a first fluorescent signal at a first intensity detectable in a first optical detection channel and the second detectable signal is the first fluorescent signal at a second intensity different from the first intensity detectable in the first optical detection channel.

13. The method of claim 1, wherein the non-target nucleic acid is an allele of a gene, and optionally the gene is selected from the group consisting of EGFR, NRAS, BRAF, KRAS, ESR1, and PIK3CA.

14. The method of claim 13, wherein the gene is EGFR, and the plurality of variants comprises: V769_D770insASV; S768_D770dup; D770_N771insG; N771_H773dup; P772_H773dup; H773_V774insNPH; H773dup; E746_A750del; L747_P753delinsS; L747_E749del; L747_P753delinQ; L747_P750delinsP; L747_A750delinsP; an amino acid substitution at G719, optionally G719A, G719S, or G719C; an amino acid substitution at T790, optionally T790M; an amino acid substitution at C797, optionally C797S; a deletion or insertion in exon 18 of EGFR; a deletion or insertion in exon 19 of EGFR; a deletion or insertion in exon 20 of EGFR; an amino acid substitution at L861, optionally L861Q; or an amino acid substitution at L858, optionally L858R.

15. The method of claim 13, wherein the gene is BRAF, and optionally wherein the plurality of variants comprises a BRAF V600 amino acid substitution, optionally V600E.

16. The method of claim 13, wherein the gene is KRAS, and optionally wherein the plurality of variants comprises a KRAS G12 amino acid substitution, optionally G12C.

17. The method of claim 10, wherein the non-target nucleic acid is an allele of a gene, and optionally the gene is selected from the group consisting of EGFR, NRAS, BRAF, KRAS, ESR1, and PIK3CA.

18. The method of claim 17, wherein the gene is EGFR, and the plurality of variants comprises: V769_D770insASV; S768_D770dup; D770_N771insG; N771_H773dup; P772_H773dup; H773_V774insNPH; H773dup; E746_A750del; L747_P753delinsS; L747_E749del; L747_P753delinQ; L747_P750delinsP; L747_A750delinsP; an amino acid substitution at G719, optionally G719A, G719S, or G719C; an amino acid substitution at T790, optionally T790M; an amino acid substitution at C797, optionally C797S; a deletion or insertion in exon 18 of EGFR; a deletion or insertion in exon 19 of EGFR; a deletion or insertion in exon 20 of EGFR; an amino acid substitution at L861, optionally L861Q; or an amino acid substitution at L858, optionally L858R.

19. The method of claim 17, wherein the gene is BRAF, and optionally wherein the plurality of variants comprises a BRAF V600 amino acid substitution, optionally V600E.

20. The method of claim 17, wherein the gene is KRAS, and optionally wherein the plurality of variants comprises a KRAS G12 amino acid substitution, optionally G12C.
